SEQUENCE LISTING (18 sequences)

SEQ ID NO: 1 - DNA, 34 bp, Homo sapiens
  source 1..34: mol_type = genomic DNA
ttggataacctagaaaaaaaacaaattactggaa

SEQ ID NO: 2 - DNA, 30 bp, Homo sapiens
  source 1..30: mol_type = genomic DNA
ggataacctagaaaaaaaaaaacaaattac

SEQ ID NO: 3 - DNA, 30 bp, Homo sapiens
  source 1..30: mol_type = genomic DNA
ggataacctagaaaaaaaaaacaaattact

SEQ ID NO: 4 - DNA, 30 bp, Homo sapiens
  source 1..30: mol_type = genomic DNA
ggataacctagaaaaaaaaacaaattactg

SEQ ID NO: 5 - DNA, 30 bp, Homo sapiens
  source 1..30: mol_type = genomic DNA
ggataacctagaaaaaaaacaaattactgg

SEQ ID NO: 6 - DNA, 30 bp, Homo sapiens
  source 1..30: mol_type = genomic DNA
ggataacctagaaaaaaacaaattactgga

SEQ ID NO: 7 - DNA, 30 bp, Homo sapiens
  source 1..30: mol_type = genomic DNA
ggataacctagaaaaaacaaattactggaa

SEQ ID NO: 8 - DNA, 30 bp, Homo sapiens
  source 1..30: mol_type = genomic DNA
ggataacctagaaaaacaaattactggaaa

SEQ ID NO: 9 - DNA, 17 bp, Homo sapiens
  source 1..17: mol_type = genomic DNA
atcatcatcatcatcat

SEQ ID NO: 10 - DNA, 24 bp, synthetic construct
  source 1..24: mol_type = other DNA
  variation 4..19
acttgaatcatcatcatcctccta

SEQ ID NO: 11 - DNA, 24 bp, synthetic construct
  source 1..24: mol_type = other DNA
  variation 5..20
acttgaatcatcatcatcctccta

SEQ ID NO: 12 - DNA, 24 bp, synthetic construct
  source 1..24: mol_type = other DNA
  S_region 6..21
acttgaatcatcatcatcctccta

SEQ ID NO: 13 - DNA, 26 bp, synthetic construct
  source 1..26: mol_type = other DNA
cgtagctgaaaaaaaaacgtacgtag

SEQ ID NO: 14 - DNA, 25 bp, synthetic construct
  source 1..25: mol_type = other DNA
cgtagctgaaaaaaaacgtacgtag

SEQ ID NO: 15 - DNA, 24 bp, synthetic construct
  source 1..24: mol_type = other DNA
cgtagctgaaaaaaacgtacgtag

SEQ ID NO: 16 - DNA, 23 bp, synthetic construct
  source 1..23: mol_type = other DNA
cgtagctgaaaaaacgtacgtag

SEQ ID NO: 17 - DNA, 22 bp, synthetic construct
  source 1..22: mol_type = other DNA
cgtagctgaaaaacgtacgtag

SEQ ID NO: 18 - DNA, 13 bp, Homo sapiens
  source 1..13: mol_type = genomic DNA
ttggataacctag

(19) United States
(12) Patent Application Publication
(10) Pub. No.: US 2023/0274794 A1
(43) Pub. Date: Aug. 31, 2023

(54) MULTICLASS CLASSIFICATION MODEL FOR STRATIFYING PATIENTS AMONG MULTIPLE CANCER TYPES BASED ON ANALYSIS OF GENETIC INFORMATION AND SYSTEMS FOR IMPLEMENTING THE SAME

(71) Applicant: AIONCO, Inc., Menlo Park, CA (US)

(72) Inventors: Cheuk Ying Tang, Cupertino, CA (US); Victor Solovyev, San Francisco, CA (US); Gene Lee, Millbrae, CA (US)

(21) Appl. No.: 18/091,331

(22) Filed: Dec. 29, 2022

Related U.S. Application Data
(60) Provisional application No. 63/294,763, filed on Dec. 29, 2021.
(60) Provisional application No. 63/294,836, filed on Dec. 29, 2021.

(51) Int. Cl. G16B 30/20; G16H 70/60; G16B 40/20; G06N 20/00; G16H 50/20; G16H 50/70

(52) U.S. Cl. CPC G16B 30/20; G16H 70/60; G16B 40/20; G06N 20/00; G16H 50/20; G16H 50/70

(57) ABSTRACT

Introduced here is an approach to training a machine learning model to classify a patient amongst multiple cancer types using sets of locations that indicate where mutations typically occur for those multiple cancer types. Upon being applied to genetic information associated with a patient whose health state is unknown, the machine learning model can produce, as input, values that indicate the likelihood of the patient having each of the multiple cancer types. Also introduced here is an approach in which diagnoses are predicted in an improved manner through the application of different models in “tiers” or “stages.” The approach may involve applying a set of multiple models to the genetic information of an individual in order to ascertain the health of the individual, and each of the multiple models can be used to indicate whether the next model in the set should be applied.

### CROSS-REFERENCE TO RELATED APPLICATIONS

This application claims priority to U.S. Provisional Application No. 63/294,763, titled “Multiclass Classification Model for Identifying Cancer of Different Types Through Analysis of Genetic Information” and filed on Dec. 29, 2021, and U.S. Provisional Application No. 63/294,836, titled “Multitier Classification for Comprehensive Determination of Cancer Presence and Type Based on Analysis of Genetic Information” and filed on Dec. 29, 2021, each of which is incorporated by reference herein in its entirety.

### REFERENCE TO SEQUENCE LISTING

This application contains an ST.26 compliant Sequence Listing, which is submitted concurrently in xml format via EFS-Web or Patent Center and is hereby incorporated by reference in its entirety. The .xml copy, created on Mar. 27, 2023, is named 145289.8003.US01 Sequence Listing.xml and is 18 KB in size.

### TECHNICAL FIELD

Various implementations concern computer programs and associated computer-implemented techniques for processing sequenced information, such as text-based representations of genetic information, for training of machine learning models.

### BACKGROUND

Genes are pieces of deoxyribonucleic acid (DNA) inside cells that indicate how to make the proteins that the human body needs to function. At a high level, DNA serves as the genetic “blueprint” that governs operation of each cell. Genes can not only affect inherited traits that are passed from a parent to a child, but can also affect whether a person is likely to develop diseases like cancer. Changes in genes—also called “mutations”—play an important role in the physiological conditions of the human body, such as in the development of cancer. Accordingly, genetic testing may be leveraged to detect such physiological conditions or likely onsets thereof.

The term “genetic testing” may be used to refer to the process by which the genes or portions of genes of a person are examined to identify mutations. There are many types of genetic tests, and new genetic tests are being developed at a rapid pace. While genetic testing can be employed in various contexts, it may be used to detect mutations that are known to be associated with cancer.

Genetic testing could also be employed as a means for addressing or treating the physiological condition. For example, after a person has been diagnosed with cancer, a healthcare professional may examine a sample of cells to look for changes in the genes to track the progression of the cancer, the efficacy of the treatment, etc. These changes may be indicative of the health of the person (and, more specifically, progression or regression of the cancer). Insights derived through genetic testing may provide information on the prognosis, for example, by indicating whether treatment has been helpful in addressing the mutation.

Implementing computing technologies for the genetic testing may yield valuable insights. For example, artificial intelligence (AI) and machine learning (ML) may be leveraged to analyze DNA information for detecting and/or addressing cancers or potential onset of cancers. However, the magnitude of the DNA information, large number of potential mutations, and large number of samples—among other factors—often negatively impact the effectiveness, accuracy, and practicality in leveraging such computing technologies for the genetic testing.

### BRIEF DESCRIPTION OF THE DRAWINGS

FIGS. 1A (SEQ ID NO:18) and 1B show example operating environments of a computing system including a genetic information processing system in accordance with one or more implementations of the present technology.

FIG. 2 shows an example data processing format for the genetic information processing system in accordance with one or more implementations of the present technology.

FIGS. 3A and 3B (SEQ ID NO:9) show examples of unique segments and refinements thereof in accordance with one or more implementations of the present technology.

FIG. 4 (SEQ ID NO:1) shows example expected phrases in accordance with one or more implementations of the present technology.

FIG. 5 (SEQ ID NO:2-SEQ ID NO:8) shows example derived phrases in accordance with one or more implementations of the present technology.

FIG. 6 shows an example analysis template in accordance with one or more implementations of the present technology.

FIG. 7 shows an example control flow diagram illustrating the functions of the processing system in accordance with one or more implementations of the present technology.

FIG. 8 shows a flow chart of a method for processing and refining DNA-based text data for cancer analysis in accordance with one or more implementations of the present technology.

FIG. 9 illustrates how the computing system can flexibly search for TR sequences (SEQ ID NO:13-SEQ ID NO:17) with different indel mutations in expected phrases in accordance with one or more implementations of the present technology.

FIG. 10 includes a flow chart of a method for training a multiclass model to stratify patients among multiple cancer types based on an analysis of genetic information.

FIG. 11 includes a flow chart of a method for applying a multiclass model that has been trained to stratify patients among multiple cancer types based on an analysis of genetic information associated with those patients.

FIG. 12 includes a chart illustrating a matrix of likelihood values output by a multiclass model upon being applied to genetic information associated with cancerous samples taken from patients known to have cancer.

FIG. 13 includes a flow chart of a method for grouping together different cancer types based on the likelihood values produced by a multiclass classification model as output.

FIG. 14 includes another example data processing format for the processing system in accordance with one or more implementations of the present technology.

FIG. 15 includes a flow chart of a method for training a binary classification model to identify the presence of cancer based on an analysis of genetic information.

FIG. 16 includes a flow chart of a method for training a binary classification model to determine whether an individual is healthy based on an analysis of genetic information.

FIG. 17 includes a flow chart of a method for applying a model set that includes at least two models.

FIG. 18 is a block diagram illustrating an example of a computing system in accordance with one or more implementations of the present technology.

Various features of the technology described herein will become more apparent to those skilled in the art from a study of the Detailed Description in conjunction with the drawings. Various implementations are depicted in the drawings for the purpose of illustration. However, those skilled in the art will recognize that alternative implementations may be employed without departing from the principles of the technology. Accordingly, although specific implementations are shown in the drawings, the technology is amenable to various modifications.

### DETAILED DESCRIPTION

Genetic testing may be beneficial for diagnosing and treating cancer. For example, identifying mutations that are indicative of cancer can help (1) healthcare professionals make appropriate decisions, (2) researchers direct their investigations, and (3) developers design better therapies, particularly through precision medicine. However, discovering these mutations tends to be difficult, especially as the number of cancers of interest (and thus, corresponding data) increases. Note that the term “mutation,” as used herein, may be used to refer to any change in a DNA sequence. Mutations may not only occur in genes but also intergenic regions and non-coding regions.

While computer-aided detection (CADe) processing system and computer-aided diagnostic (CADx) processing systems may be used to analyze data obtained through genetic testing, conventional approaches still face several drawbacks, however.

One issue is that these processing systems often struggle to distinguish between the different types of cancer. Assume, for example, that a processing system is programmed to examine nucleotides at different locations to identify mutations that are indicative of two different cancers. The first cancer—referred to as “Cancer A”— can correspond to a first set of locations at which to search for mutations, and the second cancer—referred to as “Cancer B”— can correspond to a second set of locations at which to search for mutations. The first and second sets of locations can be used as a diagnostic mechanism, either directly (e.g., for establishing whether a patient has Cancer A or Cancer B) or indirectly (e.g., for training a machine learning model to predict presence of Cancer A or Cancer B).

By examining the nucleotides existing at the first and second sets of locations in genetic information corresponding to a patient whose health state is unknown, the processing system can identify mutations that are indicative of Cancer A and Cancer B, respectively. However, despite being able to identify mutations indicative of Cancer A and Cancer B, the processing system may struggle to distinguish between these cancers with accuracy.

There may be several reasons for this. One reason is that the processing system may struggle to establish whether a mutation is more likely to be indicative of Cancer A or Cancer B if the mutation is found at a given location that is identical or similar to a first location included in the first set and a second location included in the second set. Simply put, if a mutation is discovered at a location that is included in the first set and second set—or similar to a location that is included in the first set and second set—the processing system may not have the context necessary to establish whether the mutation is more likely to be indicative of Cancer A or Cancer B. Another reason is that most processing systems are designed, programmed, or trained to identify mutations that are indicative of a single type of cancer. If a processing system is designed to only identify mutations that are indicative of Cancer A, then the processing system will not only miss mutations that are indicative of Cancer B but will also be unaware if a mutation is more likely to be indicative of Cancer B than Cancer A.

One approach to addressing these issues involves the sequential or simultaneous application of multiple machine learning models (or simply “models”), each of which is developed and trained to identify mutations that are indicative of a different cancer type. However, separately testing for different types of cancers results in significant consumption of computational resources, which can be problematic if the processing system is tasked with reviewing the genetic information of tens, hundreds, or thousands of patients. In other words, even if a processing system is able to comprehensively analyze the genetic information of a single patient, reviewing the genetic information of tens, hundreds, or thousands of patients during actual deployment becomes impractical due to processing delays and inaccuracies. Further, tasking a processing system with reviewing the genetic information of tens, hundreds, or thousands of patients may simply be infeasible due to the computational resources that would be necessary. Similar issues can plague development, namely, developing multiple model for multiple cancer types can be problematic due to the volume of genetic information that is needed for training purposes, especially since some cancer types may be associated with hundreds or thousands of molecular sites at which to search for mutations. Plus, separately analyzing for different cancers fails to offer any insights that can be gained through the relative comparison of different cancer types. As further discussed below, some insights can only be obtained by considering outputs related to different cancer types together.

Introduced here is an approach that can be implemented by a computing system to predict disease onset and/or diagnose disease presence in an improved manner. In the present disclosure, several different types of models are discussed. One of these models is a multiclass classification model (also referred to as a “multiclass model”) that is designed and then trained to simultaneously test for multiple cancer types and additionally identify non-cancerous or “healthy” inputs through analysis of genetic information. At a high level, the multiclass model can determine, through analysis of genetic information corresponding to an individual, the likelihood that the individual does not have cancer, or in the alternative, has one of multiple cancer types.

Implementations of the technology described in the present disclosure can involve the computing system processing genetic information as relatively simple computer-readable data, such as text strings—simpler in comparison to, for example, digital images. Using textual representations of genetic information, the computing system can identify specific patterns, such as unique segments of repeated characters (e.g., tandem repeats (TRs) corresponding to sequences of two or more DNA bases that are repeated numerous times in a head-to-tail manner on a chromosome), phrases surrounding the unique segments, and derivations thereof that are indicative of mutations, used to analyze nucleic acid sequences (or simply “sequences”). In some implementations, the computing system can focus on the unique phrases or derivations thereof in characterizing and/or recognizing multiple types of cancer. In some implementations, the computing system can select features from the unique phrases or derivations thereof and may ignore other portions of the larger textual representation of the sequence, thereby reducing the overall computations needed for developing, training, or applying a model or some other ML-based mechanism.

As further discussed below, a computing system can identify locations at which mutations may be indicative of the multiple cancer types and then apply the multiclass model to the genetic information corresponding to these locations. In some implementations, the multiclass model may be applied by the computing system as part of a multi-model schema. The multi-model schema may be called the “model set,” “model suite,” or “model ensemble” that is applied by the computing system to ascertain the health of individuals. The model set may include (i) a first model that is designed and trained to produce an output that indicates whether the individual is healthy, (ii) a second model that is designed and trained to produce an output that indicates whether the individual has cancer, or (iii) the multiclass model that may be referred to as the “third model” for simplicity. Accordingly, the terms “third model” and “multiclass model” may be used interchangeably.

As further discussed below, the model set could include different combinations of these models, as well as other models not described herein. For example, the model set could include the first and third models that are applied in sequence, such that the third model is applied only if the output produced by the first model indicates that the individual is not healthy. As another example, the model set could include the second and third models that are applied in sequence, such that the third model is applied only if the output produced by the second model indicates that the individual has cancer. As another example, the model set could include the first and second models that are applied in sequence, such that the second model is applied only if the output produced by the first model indicates that the individual is not healthy. As another example, the model set could include the first, second, and third models. In implementations where the model set includes all three models, the second model may only be applied if the output produced by the first model indicates that the individual is not healthy, and the third model may only be applied if the output produced by the second model indicates that the individual has cancer.

In some implementations, aspects of the first, second, and third models may be incorporated into a single “superset” model that when applied to genetic information corresponding to an individual, acts in a manner comparable to aforementioned model set. At a high level, the superset model may be representative of a multiclass model that produces outputs indicative of proposed classifications for different sets of classes. As an example, the superset model may produce a first output that indicates whether the individual is healthy or not healthy, a second output that indicates whether the individual has cancer or no cancer, and a third output that indicates which cancer types, if any, are most likely. As further discussed below, the third output may include a series of values, each of which indicates the likelihood that the individual has a corresponding cancer type.

As further discussed below, the model set can be applied to genetic information derived from samples that are not cancer specific. Examples of non-cancer-specific samples include blood samples acquired via liquid biopsy, blood samples with floating DNA acquired via blood draw, and the like. Blood samples can include DNA that is freely floating in the bloodstream, and the genetic information to be analyzed can be derived from the “floating DNA.” Moreover, the model set may be applied to genetic information derived from patients that do not have cancer or do not know they have cancer. Accordingly, the model set may be configured to consider the possibility that the analyzed genetic information does not include any cancerous markers along with detecting multiple types of cancer. In other words, the model set can be designed and trained to detect whether a non-cancer-specific sample includes any indicators of cancer, and when the non-cancer-specific sample includes such indicators, the specific cancer type(s) corresponding to the indicator(s). As a result, the computing system can comprehensively test the input—namely, genetic information corresponding to sample associated with a patient whose health state is unknown—without first assuming a health state, such as in contrast to assuming that the patient has cancer and then testing for a specific type. Thus, the model set can increase the overall accuracy (e.g., by reducing false positive outcomes or by stopping propagation of preceding diagnosis errors) of the test by removing one or more assumptions (e.g., that the patient is either healthy or unhealthy, or that the patient has cancer or does not have cancer) and conducting a test that comprehensively accounts for the additional possibilities that would otherwise be removed via the assumptions. Moreover, by specifically targeting locations in the genetic information for analysis and reducing the number of locations at which to search for mutations, the computing system can conduct the comprehensive analysis in a practical and efficient manner.

In some implementations, the model set is applied in such a manner that the computing system initially detects whether the genetic information corresponding to a sample includes cancerous indicators and then analyzes for the specific type of cancer based on finding cancerous indicators. This may be referred to as the “sequential approach” to determining the health state of the patient. In other implementations, the model set is applied in such a manner that the computing system simultaneously analyzes the genetic information corresponding to the sample for the above-described possible outcomes.

While implementation of the approach—whether performed simultaneously or sequentially—may result in improvements across different aspects of mutation discovery, there are several notable improvements worth mentioning.

While the multiclass model may be able to independently predict the likelihood of multiple cancer types, its application to genetic information may be comparatively “costly” in terms of computational resources. Advantageously, the approach may involve the sequential application of multiple models—including the multiclass model—so that these computational resources are consumed only when an individual has already been determined (e.g., based on the output produced by the first model or second model) to possibly have cancer. Simply put, computational resources may be conserved if the output produced by the first model indicates that the individual is healthy or the output produced by the second model indicates that the individual does not have cancer.

Another benefit is that appropriate diagnoses—whether positive or negative—can be determined in a timelier manner. Because model set can be applied by the computing system sequentially, individuals that are determined to not have cancer can be classified as “healthy” and then removed from the diagnostic flow, such that the multiclass model is not implemented for those individuals. This can allow healthy patients to be screened from the diagnostic flow in an effective manner. Moreover, this can allow healthcare professionals to focus their time on unhealthy patients who are more likely to need treatment. Note that the term “positive diagnosis” may be used to refer to a scenario where an individual is diagnosed as having a given cancer type, while the term “negative diagnosis” may be used to refer to a scenario where an individual is diagnosed as not having a given cancer type. Accordingly, if a computing system determines that a mutation indicative of a given cancer type is present based on an analysis of genetic information corresponding to a patient, the computing system may positively diagnose the patient with regards to the given cancer type. Meanwhile, if the computing system determines that no mutations indicative of a given cancer type are present based on an analysis of genetic information corresponding to a patient, the computing system may negatively diagnose the patient with regards to the given cancer type.

Another benefit is that the outputs produced by the multiclass model may be useful for gaining insights into the relationships between different cancer types. Assume, for example, that the multiclass model produces roughly similar values for several cancer types upon being applied to genetic information associated with a patient. In such a scenario, these roughly similar values may be analyzed separately and in combination. For example, the several cancer types—in combination—may be used to narrow the cancer experienced by the patient to a physiological region that corresponds to the several cancer types. As another example, if the several cancer types are commonly discovered through a shared testing method, then an appropriate “next step” can be determined based on the shared testing method. For instance, the shared testing method may be recommended such that results can be obtained for some or all of the several cancer types. In sum, one of the benefits of classifying the patient among multiple cancer types using a multiclass model can be improved detectability, diagnostic efficiency, and overall treatment for patients.

Practically, the approach also allows for flexibility in its usage. As further discussed below, to train the first, second, and third models, the computing system can use data that includes genetic information associated with (i) samples taken from patients known to be cancer free, (ii) samples taken from non-cancerous regions of patients known to have cancer, and/or (iii) samples taken from cancerous regions of patients known to have cancer. These samples may be referred to as “cancer-free samples,” “non-cancerous samples,” and “cancerous samples,” respectively. As such, the computing system may use the first, second, and third models (or a superset model that includes aspects of those models) to analyze random samples that are not necessarily cancer specific. As an example, the computing system may be able to analyze liquid biopsies to provide diagnoses and, if appropriate, recommend actions such as implementing specific tests, treatment plans, and the like.

Implementations may be described in the context of instructions that are executable by a computing system for the purpose of illustration. However, those skilled in the art will recognize that aspects of the technology described herein could be implemented via hardware or firmware instead of, or in addition to, software. As an example, a computer program that is representative of a software-implemented genetic information processing platform (or simply “processing platform”) designed to process genetic information may be executed by the processor of a computing system. This computer program may interface, directly or indirectly, with hardware, firmware, or other software implemented on the computing system. Moreover, this computer program may interface, directly or indirectly, with computing devices that are communicatively connected to the computing system. One example of a computing device is a network-accessible storage medium that is managed by a healthcare entity (e.g., a hospital system or diagnostic testing facility).

### Overview of Genetic Information Processing System

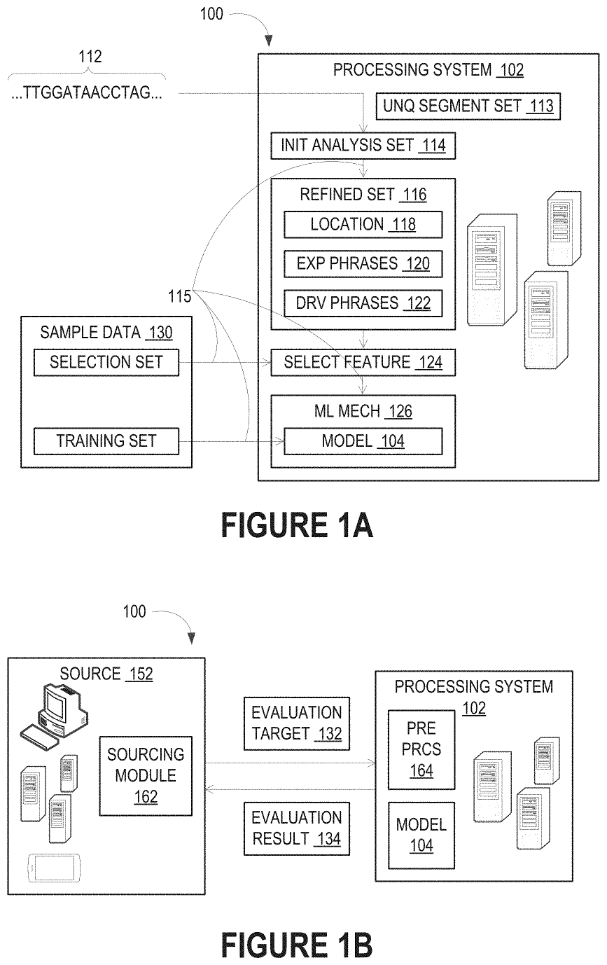

FIGS. 1A and 1B show example operating environments of a computing system 100 including a genetic information processing system 102 (or simply “processing system 102”) in accordance with one or more implementations of the present technology. The processing system 102 can include one or more computing devices, such as servers, personal devices, enterprise computing systems, distributed computing systems, cloud computing systems, and/or the like. The processing system 102 can be configured to analyze DNA information diagnosing one or more types of cancer, for evaluating development stages leading up to the onset of the one or more types of cancer, and/or for predicting a likely onset of the one or more types of cancer.

The operating environment depicted in FIG. 1A can represent a development or training environment in which the processing system 102 develops and trains an analysis mechanism, such as an ML model 104, configured to detect a presence, a progress, or a likely onset of one or more types of cancer. In developing and training the ML model 104, the processing system 102 can first identify an analysis template (e.g., specific data locations or values within reference data 112, such as the human genome or other data derived from human/patient DNA) targeted for further analysis and/or consideration.

As an illustrative example, the processing system 102 can use a text-based representation (e.g., one or more text strings) of the human DNA as the reference data 112. The processing data 102 can analyze the reference data 112 to identify specific locations and/or corresponding text sequences that can be utilized as identifiers or comparison points in subsequent processing. In some implementations, the processing system 102 can use a set of unique text segments 113 (e.g., a set of unique TRs) found or expected in the reference data 112 to generate an initial analysis set 114. The processing system 102 can generate the initial analysis set 114 by identifying expected phrases 120 that include the unique segment set 113 and/or by computing derivations thereof (e.g., derived phrases 122) that represent mutations targeted for analysis. The initial analysis set 114 and/or the unique segment set 113 can include location identifiers 118 associated with a relative location of such segments, phrases, and/or derivations within the reference data 112.

The processing system 102 can further use a refinement mechanism 115 (e.g., a software routine or a set of instructions) that further operates on the initial analysis set 114 and/or subsequent data processing. The refinement mechanism 115 can filter results of one or more data processing operations leading up to the designing and/or training of the ML model 104. The refinement mechanism 115 can generate the filtered result of the initial analysis set 114 as the refined set 116. Additionally or alternatively, the refinement mechanism 115 may be configured to filter during or after the feature selection process and/or the sample data 130.

In some implementations, the refinement mechanism 115 can process the unique segment set 113 and/or the initial analysis set 114 to generate a refined set 116. For example, the refinement mechanism 115 can be configured to remove (1) overlapping TRs from the unique segment set 113, (2) remove duplicated phrases from the initial analysis set 114, (3) filter or adjust for the sample data 130 (e.g., text-based DNA data representative of healthy individuals, cancerous tissues, and/or non-cancerous tissues collected from cancer patients) used to develop and/or train the ML model 104, and/or (4) adjust for, or filter, physiological noise or processing noise. Details regarding the derivation of the initial template and refinement thereof are described below.

For the feature selection, the processing system 102 can iteratively add or remove one or more unique locations/sequences and/or derivations from the refined set 116 and calculate a correlation or an effect of the removed datapoint on the known classifications of the sample data 130 (e.g., to accurately recognize the different categories of the sample data 130). The processing system 102 can determine a set of selected features 124 that correspond to the unique locations/sequences and derivations thereof having at least a threshold amount of effect or correlation with one or more corresponding cancer types. In other words, the processing system 102 can determine the set of features 124 including locations, sequences, mutations, or combinations thereof that are deterministic or characteristic of, or commonly occurring in, corresponding cancers. Based on the set of features 124, the processing system 102 can implement an ML mechanism 124 (e.g., a support vector machine (SVM), a random forest, neural network, etc.) to generate the ML model 104. The processing system 102 can further train the ML model 104 using training data.

Using the refined results, the processing system 102 can limit the amount of data considered or processed in subsequent analyses, such as in feature selection, model generation, model training, and/or the like. For example, the processing system 102 can use the refinement mechanism 115 to reduce the size of the unique segment set 113, thereby reducing the expected phrases 120 and the derived phrases 122 that correspond to the unique segment set 113. Also, the processing system 102 can use the refinement mechanism 115 to further reduce the size of the initial analysis set 114, such as by removing potential duplicated phrases (e.g., across expected/derived phrases at different locations). Accordingly, the processing system 102 can reduce the resource consumption through the reduced size of the refined set 116 (e.g., in comparison to the initial analysis set 114) and reduce the noises and other negative impacts generated by the overlapping/duplicative phrases. Additional sample-, process-, or physiology-based refinement can further increase the overall performance and accuracy of the resulting ML model 104.

The operating environment depicted in FIG. 1B can represent a deployment environment in which the processing system 102 applies the analysis mechanism to detect a presence, a progress, and/or a likely onset of one or more types of cancer from an evaluation target 132 (e.g., a text-based form of patient DNA data). The processing system 102 can generate an evaluation result 134 based on testing the evaluation target 132 with the ML model 104. The processing system 102 can generate the evaluation result 134 that represents a cancer diagnosis or a cancer signal. For example, the evaluation result 134 can represent a determination that the patient has cancer, a stage (e.g., clinically recognized stages 1-4) of the onset cancer, a progress state before, or leading up to, an onset of caner, a likelihood of developing cancer within a predetermined period, an identification of the type of cancer, or a combination thereof.

As an illustrative example, the processing system 102 can include a sourcing device 152 that provides the evaluation target 132 and/or receives the evaluation result 134. The sourcing device 152 can be operated by a patient submitting the evaluation target 132, a healthcare service provider associated with the patient, an insurance company, or the like. Some examples of the sourcing device 152 can include a personal device (e.g., a personal computer or a mobile computing device, such as a wearable device, smart phone, or tablet), a workstation, an enterprise device, etc.

In some implementations, the processing system 102 can include a sourcing module 162 that operates on the sourcing device 152. The sourcing module 162 can include a device, circuit, or a software module (e.g., a codec, application program, or the like) that generates or pre-processes the evaluation target 132. For example, the sourcing module 162 can include a homomorphic encoder that encrypts and prevents unauthorized access to the patient data. The evaluation target 132 can include the homomorphically encoded data that can be processed at the processing system 102 without fully decrypting and recovering the patient data. In other words, the processing system 102 can apply the ML model 104 that is configured to process or perform computations on the encrypted data.

The processing system 102 can include a pre-processing module 164 that conditions the evaluation target 132 for and/or during application of the ML model 104. For example, the pre-processing module 164 can include a device, circuit, or a software module (e.g., a codec, application program, or the like) that removes biases or noises introduced before receiving the evaluation target 132 and/or during the processing (e.g., bootstrapping module to remove noise or other uncertainties introduced by processing encrypted data) of the evaluation target 132.

### Data Processing Formats

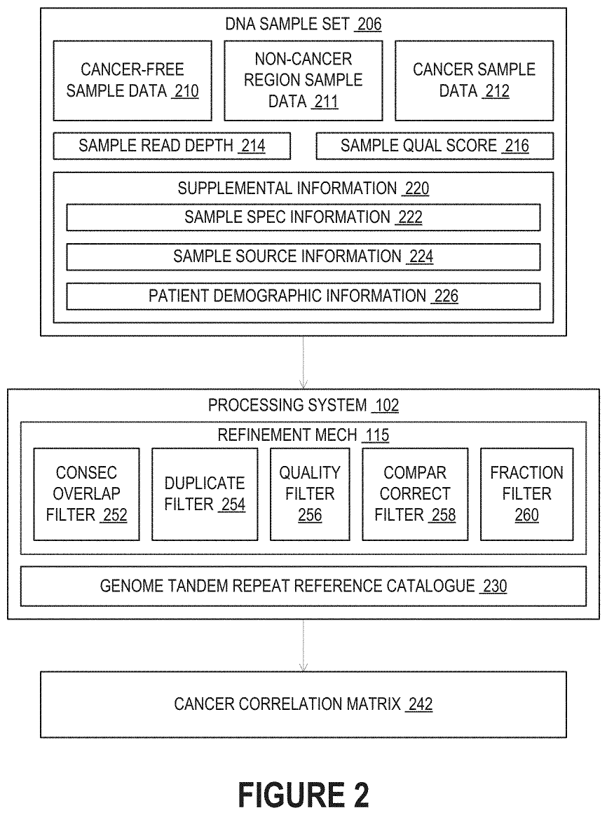

In developing and training the ML model 104 and/or deploying the ML model 104, the processing system 102 can utilize a variety of data processing formats (e.g., data structures, organizations, inputs and outputs, or the like). FIG. 2 shows an example data processing format for the processing system 102 in accordance with one or more implementations of the present technology. The processing system 102 receive and process a DNA sample set 206 (e.g., an instance of the reference data 112 and/or sample data 130 illustrated in FIG. 1A) having one or more of the formats or subfields illustrated in FIG. 2. Moreover, the processing system 102 can generate the initial analysis set 114 (FIG. 1A) and the refined set 116 (FIG. 1A) using one or more detailed example aspects illustrated in FIG. 2.

As an illustrative example, the DNA sample set 206 can include DNA data (e.g., representative of a set of sequenced DNA information) corresponding to different known categories. Examples of the DNA sample set 206 can include genetic information (e.g., text-based representations) derived or extracted from human bodies, such as from tissue extracted during a biopsy or from cell-free DNA (e.g., DNA that is not encapsulated within a cell) in bodily fluids. The DNA sample set 206 can include DNA data collected from volunteers or participating patients having medically confirmed diagnoses and/or from public or private databases.

The DNA sample set 206 can include data collected from different types and/or categories of samples, such as cancer-free samples (cancer-free sample data 210), samples taken from non-cancerous regions (non-cancer region sample data 211), and/or cancerous samples (cancer sample data 212). The cancer-free sample data 210 (or simply “cancer-free data”) can represent text-based DNA data corresponding to samples collected from patients confirmed/diagnosed to be cancer free. The non-cancer region sample data 211 (also called “non-regional data”) can represent text-based DNA data corresponding to samples collected from non-cancerous regions (e.g., white blood cells or leukocytes) of patients confirmed/diagnosed to have one or more types of cancer. The cancer sample data 212 (also called “cancer-specific data”) can represent text-based DNA data corresponding to samples (e.g., tumor biopsies, liquid biopsies, etc.) collected from cancerous regions or tumors confirmed/diagnosed to be a specified type of cancer. The DNA sample set 206 can include information (e.g., the non-regional data 211 and/or the cancer-specific data 212) corresponding to one or more types of cancers (e.g., breast cancer, lung cancer, colon cancer, and/or the like).

The DNA sample set 206 can further include descriptions regarding a strength or a trustworthiness of the data. For example, the DNA sample set 206 can include a sample read depth 214 and/or a sample quality score 216. The sample read depth 214 can represent a number of times that a given nucleotide in the genome (e.g., certain text string/portion) was detected in a sample. The sample read depth 214 may correspond to a sequencing depth associated with processing fragmented sections of the genome within a tissue sample. The sample quality score 216 can represent a quality of identification of the nucleobases generated by DNA sequencing. In some implementations, the sample quality score 216 can include a Phred quality score.

The DNA sample set 206 can also include supplemental information 220 that describes other aspects of the sample or the source of the data. For example, the supplemental information 220 can include information such as sample specification information 222 (or simply “specification information”), sample source information 224 (or simply “source information”), patient demographic information 226, or a combination thereof.

The specification information 222 can include technical information or specifications about the sequenced DNA associated with the DNA sample set 206. For example, the specification information 222 can include information about the locations 118 (FIG. 1A) within the genome to which the DNA fragments correspond, such as intron and exon regions, specific genes, or chromosomes. Also, the specification information 222 can describe, for example, (1) the process, methods, and instrumentation used to extract and sequence the genetic material, (2) the number of sequencing reads for each sample, or a combination thereof.

The source information 224 can include details regarding the source and/or the categorization of the sample. For example, the source information 224 can include information about the cancer type, the stage of cancer development, the organ or tissue from which the sample was extracted, or a combination thereof.

The patient demographic information 226 can include demographic details about the patient from which the sample was taken. For example, the patient demographic information 226 can include the age, the gender, the ethnicity, the geographic location of where the patient resides/visited, the duration of residence/visitation, predispositions for genetic disorders or cancer development, family history, or a combination thereof.

The processing system 102 can analyze the DNA sample set 206 using the mutation analysis mechanism. Accordingly, the processing system 102 can identify mutations or mutation patterns in specific DNA sequences that can be used as markers to determine the existence, the progress, and/or the developing stages of a particular form of cancer. To identify the relevant mutations, the processing system 102 can detect a set of targeted locations or text patterns (e.g., according to the TRs) within the reference genomes.

The processing system 102 can generate and/or utilize a genome tandem repeat reference catalogue 230 that represents a catalogue or a collection of uniquely identifiable TRs in the human genome. As an example, the genome tandem repeat reference catalogue 230 can be based on a reference human genome (e.g., the reference data 112), such as the GRCh38 reference genome. The uniquely identifiable TRs can include DNA sequences having therein a series of multiple instances of directly adjacent identical repeating nucleotide units or base patterns, such as microsatellite DNA sequences. The base patterns can have a predetermined length, such as one for a repetition of one letter or monomer (e.g., ‘AAAA’) or greater (e.g., three for tetramers, such as ‘ACT’). Such uniquely identifiable TRs can serve as reference sequences (e.g., reference locations within the human genome) or markers for evaluating the DNA sample set 206. Since the DNA sample set 206 may correspond to incomplete DNA fragments, the unique TRs found within the fragments may be used to map the DNA information to the human genome.

The processing system 102 can use the genome tandem repeat reference catalogue 230 to compute the initial analysis set 114. For example, the processing system 102 can use the unique TRs identified in the genome tandem repeat reference catalogue 230 to generate derived strings that represent potential mutations. In some implementations, the processing system 102 can identify text characters preceding and/or following each unique TR and derive the mutation strings that represent one or more types of mutations (e.g., insertion-deletion mutations—also called “indel mutations” or “indels”). Details regarding the initial analysis set 114 (e.g., strings with flanking characters and/or mutation strings) are described below.

The processing system 102 can compare the mutations at the targeted locations/sequences across the different types of DNA sample set 206. Based on the comparison, the processing system 102 can compute a correlation between, or a likely contribution of, the mutations at the targeted locations/sequences and the development of cancer. Accordingly, the processing system 102 may generate a cancer correlation matrix 242 that correlates identified tumorous sequences or text-based patterns to specific types of cancer. For example, the cancer correlation matrix 242 can be an index that includes multiple instances of the uniquely identifiable TRs in the genome TR reference catalogue 230 that, when found to be tumorous, indicate the existence of a particular form of cancer or indicate the possibility that a particular form of cancer will develop.

The processing system 102 can perform the feature selection using the cancer correlation matrix 242, such as by retaining the locations/sequences and/or derived mutation patterns having at least a predetermined degree of correlation to one or more corresponding types of cancer. Using the selected features, the processing system 102 can develop and train the ML model 104 configured to detect, predict, and/or evaluate development or onset of cancer.

In some implementations, the processing system 102 can further use the refinement mechanism 115 to generate the refined set 116 (FIG. 1A). The refinement mechanism 115 may include one or more filters to enhance the genome TR reference catalogue 230, the initial analysis set 114, and/or corresponding features, such as by removing or adjusting one or more erroneous or unnecessary sequences. For example, the refinement mechanism 115 can include: (1) a consecutive overlap filter 252 configured to remove consecutive or overlapping sequences (e.g., unique TRs) that effectively point to the same location, (2) a duplicate filter 254 configured to remove duplicate sequences, such as between mutation strings at different locations, (3) a quality filter 256 configured to remove/adjust for input sample data, such as based on quality and/or input depth, (4) a comparison correction filter 258 configured to remove computational noise or errors, (5) a physiology-based filter, such as a fraction filter 260, configured to remove or adjust for physiological features and/or collection-based features that interfere with the data processing, or a combination thereof. Details regarding the refinement mechanism 115 is described below.

### Base Text Patterns—Segments

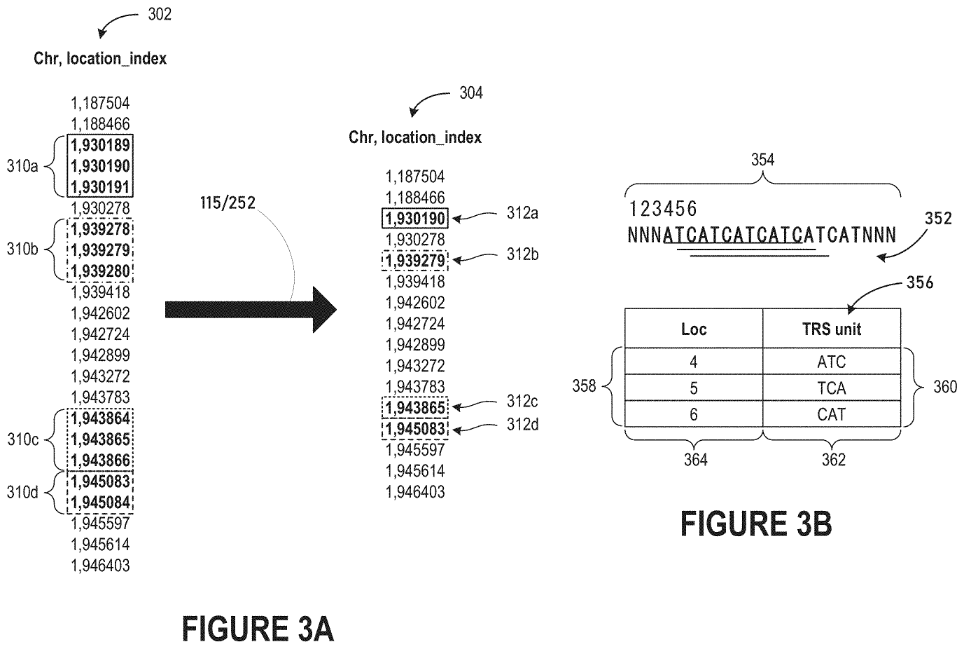

For describing further detailed aspects of the data format, FIGS. 3A and 3B show examples of unique segments (e.g., uniquely identifiable TRs within the human genome) and refinements thereof in accordance with one or more implementations of the present technology. FIG. 3A shows an initial segment set 302 and a refined segment set 304 that correspond to the unique segments 113 of FIG. 1. FIG. 3B illustrates example overlaps 352 in the initial segment set 302. Referring to FIGS. 3A and 3B together, the processing system 102 can use the refinement mechanism 115 (e.g., the consecutive overlap filter 252) to remove the overlaps 352 therein and generate the refined segment set 304.

In some implementations, the processing system 102 can generate the initial segment set 302 based on analyzing the reference data 112 (FIG. 1A) to find uniquely identifiable patterns. For example, the processing system 102 can generate the initial segment set 302 by identifying uniquely identifiable TRs within the human genome. The processing system 102 can use base or TR units (e.g., base character patterns having controllable lengths of one or more characters that are repeated) to identify the overall TR or segment having a corresponding length (e.g., two or more multiples of the TR unit length). The processing system 102 can generate the initial segment set 302 by including repeated patterns of the TRs that exceed a minimum number of base pairs. For example, the repeated TR sequence can be selected based on using the repeated base unit having the minimum number of base pairs ranging between five and eight base pairs.

In the initial segment set 302, the processing system 102 may end up including the overlaps 352 that effectively correspond to a longer and unique string segment and the corresponding location. For the example illustrated in FIG. 3B, a target sequence 354 (e.g., a sequence/combination of nucleotides, such as a portion of the DNA information) can include a uniquely identifiable segment (‘ATCATCATCATCATCAT’ (SEQ ID NO:9) having 17 characters, with 3 preceding placeholder characters and 3 succeeding placeholder characters referenced with ‘N’). The processing system 102 can identify unique segments 360 within the target sequence 354 based on identifying repeated adjacent patterns of base units 362. The length of the repeated base units 362 and/or the number of repeats may be predetermined or adjusted in generating the initial segment set 302. For the illustrated example, the targeted segment length corresponds to 12 characters or four repeats of three-letter TR units. Along with the repeated base units 362, the unique segments 360 can be identified based on corresponding segment locations 364 that identify positions (e.g., first letter positions) of the segments within the target sequence 354.

When the target sequence 354 includes a repeated pattern that exceeds the targeted segment length, one target sequence 354 can be identified as including repeats of multiple instances of the base units 356 (e.g., ‘ATC,’ ‘TCA,’ and ‘CAT’). The multiple instances of the base units 356 may correspond to shifted results of each other. As such, the multiple unique segments 360 can overlap each other and/or be sequentially shifted by one or more characters relative to each other. FIG. 3A illustrates a portion of the initial segment set 302 having overlapping location sets 310a, 310b, 310c, and 310d that correspond to such overlapping instances of the unique segments 360. However, given the nature of the overlaps, each of the overlapping location sets 310a, 310b, 310c, and 310d can effectively correspond to a single segment/location rather than the multiple separate segments/locations.

The processing system 102 can use the refinement mechanism 115 to identify and remove the overlaps 352 in the unique segments 360. In some implementations, the consecutive overlap filter 252 can be configured to ensure that the initial segment set 302 is sorted according to the segment location 358. With the sorted segments, the consecutive overlap filter 252 can identify patterns in the segment location 358 of adjacent segments within the initial segment set 302. The consecutive overlap filter 252 can be configured to identify the overlaps 352 when the segment location 358 of the adjacent segments are separated by a predetermined number (e.g., one, two, or more, a number based on the repeated unit length and/or the targeted segment length, and/or the like). Also, the consecutive overlap filter 252 can be configured to identify the overlaps 352 when the segment location 358 follows one or more pattern (e.g., consistently separated by one or two values) over two, three, or more adjacently occurring segments. The consecutive overlap filter 252 can group the two or more adjacent segments that satisfy the separation threshold/pattern as a set of the overlaps.

Additionally or alternatively, the consecutive overlap filter 252 can configured to identify the overlaps 352 when the repeated base units 356 for the adjacent segments correspond to circularly shifted values. For the example illustrated in FIG. 3B, the processing system 102 can identify that the unique segments 360 at locations 4, 5, and 6 correspond to an overlapping set since the repeated base units 356 of ‘ATC,’ ‘TCA,’ and ‘CAT’ correspond to circularly shifting a preceding unit by one character/position. The consecutive overlap filter 252 can group the two or more adjacent segments that satisfy/maintain the detected pattern in the repeated base units 356 a set of the overlaps.

After the sets of overlaps are identified, the consecutive overlap filter 252 can refine the set by reducing the number of overlapped segments. For example, the consecutive overlap filter 252 can retain one segment from each set of overlaps and remove others. In some implementations, the consecutive overlap filter 252 can be configured to select the segment according to a predetermined location, the target segment length, the repeated unit length, or a combination thereof. For example, the consecutive overlap filter 252 can be configured to select the segment positioned in the middle/center of the set. Also, the consecutive overlap filter 252 can include a predetermined equation that identifies the selection location according to the number of segments in the set, the target segment length, the repeated unit length, or a combination thereof. The selected locations can be represented as refined locations (e.g., refined locations 312a, 312b, 312c, and 312d respectively corresponding to overlapping sets 310a, 310b, 310c, and 312d) in the refined segment set 304.

### Base Text Patterns—Expected Phrases

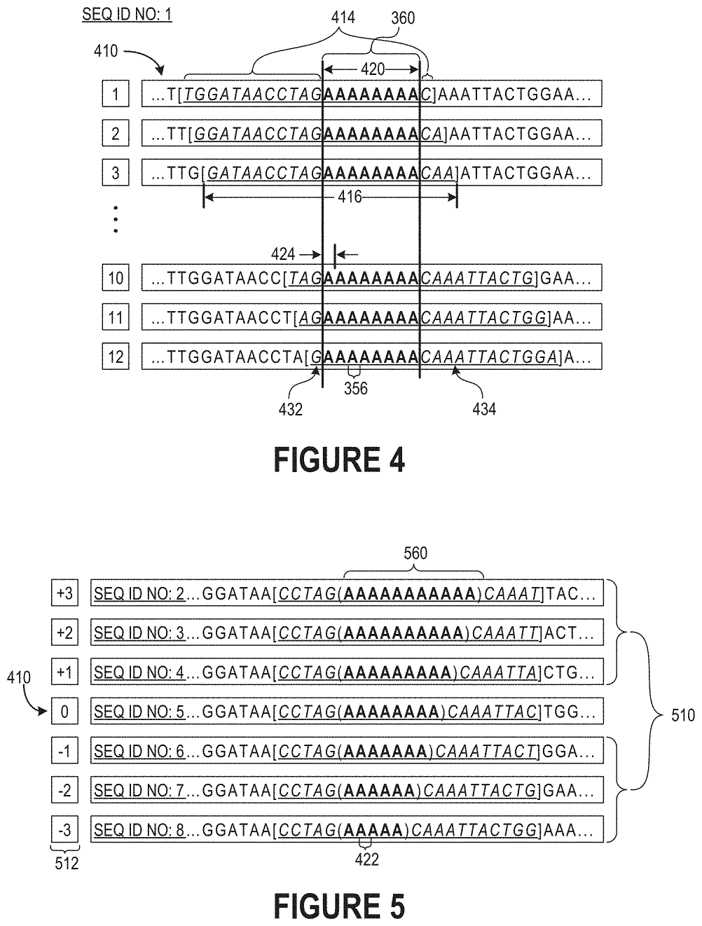

The processing system 102 can use the processed segments (e.g., the refined segment set 304) to generate phrases. FIG. 4 shows example expected phrases 410 in accordance with one or more implementations of the present technology. The expected phrases 410 can correspond to textual representations of the DNA sequences or a set of sequence variations that may be used as bases for subsequent processing/comparing, such as in deriving mutations strings and analyzing the DNA sample set 206 (FIG. 2).

For context, samples collected from patients may include fragments or portions of the overall DNA. As such, the corresponding sequenced values or the text string may include different combinations of characters. The processing system 102 (FIG. 1A) can generate the expected phrases 410 as representations of different character combinations that include the uniquely identifiable segments (e.g., the refined segment set 304 (FIG. 3A), such as the refined set of unique TRs).

Accordingly, the processing system 102 can generate the expected phrases 410 based on the refined segment set 304 instead of the initial segment set 302 (FIG. 3A). In some implementations, the processing system 102 can generate a set (illustrated as a unique sequence identifier number in FIG. 4) of the expected phrases 410 for each of the unique segments 360 (illustrated using bolded characters in FIG. 4) in the refined segment set 304.

The expected phrases 410 can have a phrase length 416 of k (e.g., generally between 10 to 50, but could be greater than 50 or fewer than 10) number of DNA base pairs or pairs of nucleobases. Each DNA base pair can be represented as a single text character (e.g., ‘A’ for adenine, ‘C’ for cytosine, ‘G’ for guanine, and ‘T’ for thymine). As such, the expected phrases 410 may also be referred to as “k-mers.”

In some implementations, as described above, the unique segments 360 can include a DNA sequence of a specified minimum length. A unique segment 360 can include a series of multiple instances of directly adjacent identical repeating nucleotide units or the repeated base units 356. For example, the unique segment 360 can include a minisatellite DNA or microsatellite DNA sequence of a specified minimum length. Accordingly, the unique segment 360 can correspond to a repeated pattern of the repeated base units 356, and the number of repetitions can correspond to a segment length 420 (e.g., the total length of, or total number of, nucleotide base pairs) for the unique segment 360. The repeated base unit 356 can have a base unit length 424 corresponding to the number of nucleotides within the repeated base unit 356 (e.g., one for a mono-nucleotide, two for a di-nucleotide, etc.).

For illustrative purposes, FIG. 4 shows a specific instance for the unique segment 360 of “AAAAAAAA,” annotated as “A8,” located at the molecular position starting at “10,513,372” on chromosome 22. In this example, the unique segment 360 includes the segment length 420 of eight base pairs with the repeated base unit 356 of one base pair (e.g., a monomer or a mono-nucleotide) ‘A.’

The processing system 102 can use the phrase length 416 (e.g., k between 10 to 50 base pairs) that has been predetermined or selected to capture targeted amount of data/characters surrounding the unique segments 360. As such, the phrase length 416 can be greater than the segment length 420, and each of the expected phrases 410 can include a set of flanking texts 414 (e.g., text-based patterns; illustrated using italics in FIG. 4) preceding and/or following the corresponding unique segment 360.

The processing system 102 can generate the expected phrases 410 in a variety of ways. As an illustrative example, the processing system 102 can use each of the unique segments 360 as an anchor for a sliding window having a length matching the phrase length 416. The processing system 102 can iteratively move the sliding window relative to the unique segment 360 and log the text captured within the window as an instance of the expected phrases 410. As such, each of the expected phrases 410 can correspond to a unique position of the sliding window relative to the unique segment 360. Also, the set of expected phrases 410 for one reference TR can include different combinations of the flanking text 414 (e.g., a combination of one or more leading characters 432 and/or one or more tailing characters 434).

The total number of base pairs in flanking text 414 can be a fixed value that is based on the phrase length 416 and the segment length 420. The number of characters in the flanking text 414 can be calculated as the difference between the phrase length 416 and the segment length 420. As an example, for one of phrases having a length of 21 base pairs and a segment length of 8 base pairs, the flanking text can include 13 base pairs.

Each of the expected phrases 410 can represent one of a number of position variant k-mers based on the flanking texts 414. The position variant k-mers can include specific numbers of base pairs in the leading flanking text 432 and tailing flanking text 434. For example, a set of the expected phrases 410 can include the same unique segment (e.g., repeated pattern of the TR) and differ from one another according to the number of base pairs included in the leading flanking text 432 and/or the tailing flanking text 434. In general, the number of base pairs included in the leading flanking text 432 and tailing flanking text 434 can vary inversely between the different instances of the position variant k-mers or expected phrases 410.

As an example, each of the expected phrases 410 illustrated in FIG. 4 has the phrase length 416 of 21 base pairs and the segment length 420 of 8 base pairs. A first expected phrase can have the leading characters 432 corresponding to 12 base pairs and the tailing character 434 corresponding to 1 base pair. A second expected phrase can have the leading characters 432 corresponding to 11 base pairs and the tailing characters 434 of 2 base pairs. The pattern can be repeated until the last expected phrase has the leading characters 432 corresponding to 1 base pair and the tailing characters 434 corresponding to 12 base pairs.

The expected phrases 410 can be grouped into sets that each correspond to a unique segment as described above. The total number of phrases or position variant k-mers (position variant total) in the grouped set can be represented as:

Position Variant Total=(Phrase length k)−(Segment length)−1.

For the example illustrated in FIG. 4, the set of expected phrases can have a position variant total of 12, representing 12 different instances of phrases corresponding to the phrase length 416 of 21 and the segment length 420 of 8.

In some implementations, the processing system 102 can use the unique instances of the TRs as the basis for generating the sets of expected phrases 410. Accordingly, each of the expected phrases 410 can also be unique since it is generated using the corresponding unique TR as a basis. The processing system 102 can use the unique expected phrases 410 to account for and identify the fragmentations likely to be included in the patient samples.

### Base Text Patterns—Derived Phrases

The processing system 102 can use the expected phrases 410 to analyzes mutations in genetic information (e.g., sequenced DNA segments), such as for detecting tumorous/cancerous DNA sequences. The expected phrases 410 can be used to detect locations within the reference genome and related mutations that are indicative of certain types of cancers or likely onset thereof. The processing system 102 can use the expected phrases 410 as basis to generate derived phrases that represent various mutations in the genetic information. The processing system 102 can use the derived phrases to recognize or detect mutations in the DNA sample set 206 (FIG. 2), the sample data 130 (FIG. 1A), or the like in developing, training, and/or deploying the ML model 104. Effectively, the processing system 102 can identify the mutation patterns indicative of certain types of cancers based on using the derived phrases to determine differences between healthy and cancerous DNA samples (e.g., between the cancer-free data 210, the non-regional data 211, and/or the cancer-specific data 212 illustrated in FIG. 2).

FIG. 5 shows example derived phrases 510 in accordance with one or more implementations of the present technology. The processing system 102 (FIG. 1A) can generate the derived phrases 510 based on adjusting the expected phrases 410 expected to a predetermined pattern. For example, for one or more or each of the expected phrases 410, the processing system 102 can generate a set of the derived phrases 510 that represent indel mutations of the corresponding expected phrase 410. In some implementations, the processing system 102 can generate the set of derived phrases 510 that correspond to a predetermined number of insertions and/or deletions in the unique segment 360 (FIG. 4) within the corresponding expected phrase 410. In other words, the set of derived phrases 510 can represent the indel variants of the sequence represented by the corresponding expected phrase 410.

The processing system 102 can generate the set of the derived phrases 510 based on adjusting (via insertion/deletion) the number of the repeated base units 356 (FIG. 4) and/or one or more characters in the unique segment 360 of the expected phrase 410. Accordingly, the processing system 102 can generate a set of derived segments 560 that correspond to indel variants of the unique segment 360.

The processing system 102 can generate the derived phrases 510 based on adding and/or adjusting the flanking text 414 (FIG. 4) around the derived segments 560 (illustrated as the bolded characters within parentheses ‘0’). In some implementations, the processing system 102 can generate the derived phrases 510 having the same phrase length 416 (FIG. 4) as the expected phrases 410. As a result, the processing system 102 can expand or reduce the coverage of the flanking text 414 according to the indel changes to the unique segment 360 (e.g., the originating pattern of TRs). With deletions, the processing system 102 can include corresponding number of new characters from the overall sequence into the flanking text 414 (FIG. 4). Similarly with additions, the processing system 102 can remove the corresponding number of characters from the flanking text 414. For illustrative purposes, FIG. 5 shows the surrounding adjustments occurring in the trailing characters 434 (FIG. 4) while maintaining the leading characters 432 (FIG. 4). However, it is understood that the processing system 102 can operate differently, such as by (1) adjusting the leading characters 432 while maintaining the trailing characters 434 and/or (2) spreading the adjustments across the leading characters 432 and the trailing characters 434 according to the number of characters in the original phrase and/or a predetermined pattern.

For the example illustrated in FIG. 5, the expected phrase 410 can correspond to the repeated TR sequence of “AAAAAAAA” or A8 beginning at position 10,513,372 on chromosome 22. The derived phrases 510 can correspond to the derived segments 560 including up to three insertions and deletions of the repeated base unit ‘A.’ In other words, the derived phrases 510 can correspond to phrases built around A5, A6, A7, A9, A10, and A11.

The number of the derived phrases 510 associated with a given expected phrase can be determined by an indel variant value 512. The indel variant value 512 can include an integer value representative of the number of insertions and deletions. The indel variant value 512 can further function as an identifier for a phrase. For example, the indel variant value ‘0’ can represent the expected phrase 410 having zero insertions/deletions. Positive indel variant values (e.g., 1, 2, 3) can represent derived phrases including corresponding number of insertions of base units or characters in the repeated TR portion. Negative indel variant values (e.g., −1, −2, −3) can represent derived phrases corresponding number of deletions of base units or characters in the repeated TR portion. For the example illustrated in FIG. 5, the indel variant values 1, 2, and 3 can represent/identify A9, A10, and A11, respectively. Also, the indel variant values −1, −2, and −3 can represent A7, A6, and A5, respectively.

For context, the processing system 102 can use the expected phrases 410 and the corresponding sets of derived phrases 510 to analyze the DNA sample set 206 and develop/test the ML model 104 (FIG. 1A). The phrases generated using the unique TR patterns can provide accurate and precise identification of corresponding sequences in the different types of health and cancerous DNA samples. In other words, the various phrases can represent the type of textual patterns or the corresponding sequences that are targeted for analyses and comparisons between the cancer-free data 210, the non-regional data 211, and/or the cancer-specific data 212. For example, the processing system 102 can use the various phrases to identify the numbers and types/locations of mutations in the cancer-related samples and absent in healthy samples. The processing system 102 can aggregate the results across multiple samples and patients to derive a pattern or a correlation between certain types of mutations and the onset of certain types of cancer.

To put things another way, the processing system 102 can identify unique patterns (e.g., the unique TR patterns and/or the corresponding expected phrases 410) that each occur once within the human genome. The unique patterns can be used to identify specific locations and portions within the human genome for various analyses. Moreover, the processing system 102 can target specific types of mutations, such as indel mutations, in developing a cancer-screening tool and/or a cancer-predicting tool. It has been found that various types of cancers can be accurately detected and progress/status of such types of cancers can be described using the expected phrases 410 and the corresponding sets of the derived phrases 510 (e.g., sequences identified using unique TR-based patterns and indel variants thereof) and without considering other aspects/mutations of the human DNA. As a result, the processing system 102 can generate the ML model 104 that can accurately detect the existence, predict a likely onset, and/or describe a progress of certain types of cancers using the various phrases. In other words, the processing system 102 can detect/predict the onset of cancer without processing the entire DNA sequence and different types of mutation patterns.

The processing system 102 can further improve the efficiency and reduce the resource consumption using the indel variant value 512. Given the downstream processing methodology, the indel variant value 512 can control the number of phrases considered in developing/training the ML model 104 and thereby affect the overall number of computations and the amount of resource consumption. When the indel variant value 512 is too high, the processing system 102 may end up analyzing a reduced or ineffective number of possible sequences. For example, as the total number of base pairs in the TR indel variant approaches the phrase length 416, the number of available derived phrases and the likely occurrence of such mutations decrease. Accordingly, in some implementations, the indel variant value 512 in the range of three to five provides sufficient coverage for varying degrees of possible insertion and deletion mutations that are indicative of one or more types of cancer. This range of values may be sufficient to provide accurate results without requiring ineffective or inefficient amount of computing resources.

Additionally, the processing system 102 can further improve the efficiency and reduce the resource consumption using the segment length 420 (e.g., the length of the uniquely identifiable TR-based pattern). It has been found that the probability of mutation occurrences decreases as the tandem repeat segment length 420 is reduced. In particular, the mutation rate for genome TR sequences with segment length 420 of fewer than five base pairs is significantly less than genome TR sequences with segment length 420 of five or more base pairs. Thus, the expected phrases 410 can be selected as the genome TR sequence with segment length 420 of five or greater.

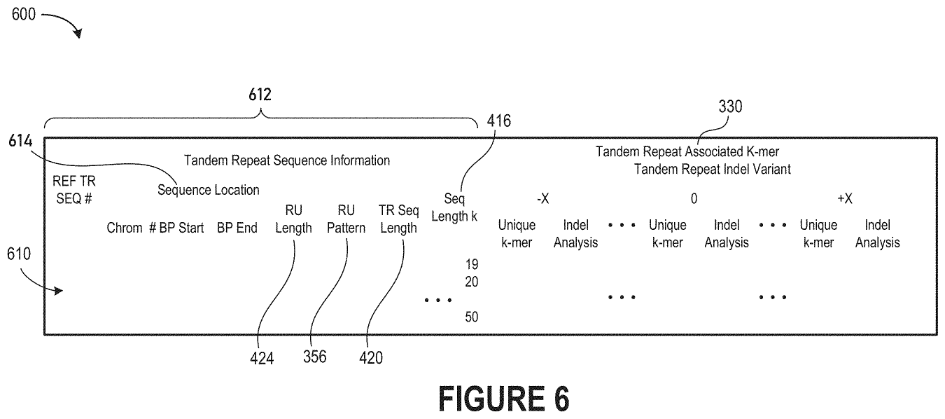

The processing system 102 can store the various phrases (e.g., the expected phrases 410 and/or the corresponding sets of the derived phrases 510) in the genome TR reference catalogue 230 (FIG. 2). FIG. 6 shows an example analysis template 600 in accordance with one or more implementations of the present technology. The processing system 102 can use the analysis template 600 to represent the various phrases and/or track the associated processing results.

In some implementations, the analysis template 600 can correspond to a format for the genome TR reference catalogue 230. The genome TR reference catalogue 230 can include catalogue entries 610 for each instance of the unique segments 360 (e.g., uniquely identifiable TR patterns or reference TR patterns). The entries 610 can include TR sequence information 612 that characterizes the unique segments 360 and/or the derived segments 560. For example, the TR sequence information 612 can include a sequence location 614, the segment length 420, the base unit length 424, the repeated base unit 356, or a combination thereof.

The sequence location 614 can identify the location of the corresponding unique segment 360 and/or expected phrase 410 within the reference genome. As an example, the sequence location 614 can be described based on the molecular location of the unique segment 360, such as (1) the chromosome on which the TR sequence is located and/or (2) the base pair numbers in the chromosome marking the beginning/ending of the TR sequence. The sequence location 614 can act as a unique identifier that distinguishes one instance of the unique segment 360 and/or the expected phrase 410 from another. For example, expected phrases 410 that share the same repeated base unit 356 and the base unit length 424 can be distinguished from one another based on the sequence location 614.

The entries 610 for each instance of the unique segment 360 can include information for one or more instances of the corresponding phrases (e.g., expected and/or derived). For example, the entries 610 can include information for the expected phrases 410 and/or the derived phrases 510 with various values for the phrase length 416. For illustrative purposes, this instance of entries 610 is shown including information for the expected phrases 410 with phrase lengths corresponding from 19 base pairs to 60 base pairs. However, it is understood that the entries 610 can include information regarding expected phrases 410 with fewer than 19 base pairs and/or greater than 60 base pairs. As another example, the entries 610 can include information that distinguishes between the expected phrases 410 and the derived phrases 510. In some implementations, the entries 610 can identify expected phrases 410 associated with a corresponding TR pattern. For instance, the TR pattern of ‘A8’ beginning at position 10,513,372 can yield 16 sequences or expected phrases 410 having the phrase length 416 of 30 base pairs.

The entries 610 can further identify the derived phrases 510 that are absent from the reference genome. For illustrative purposes, Table 1 below summarizes the derived phrases 510 having the segment length 416 of 30 base pairs for the unique segment 360 or TR pattern of ‘A8’ beginning at position 10,513,372 (annotated as '372) on chromosome 22. In this example, each of the derived phrases 510 corresponding to indel variants with the indel variant value 512 ranging from “−5” to “+5” are not found in the reference genome.

TABLE 1
Chromosome 22, ′372, “A8” Reference
TR Associated Indel Phrase Summary

| Indel Variant Value | Position Variant Total | Total That Do Not Appear |
|---|---|---|
| +5 | 16 | 16 |
| +4 | 17 | 17 |
| +3 | 18 | 18 |
| +2 | 19 | 19 |
| +1 | 20 | 20 |
| −1 | 22 | 22 |
| −2 | 23 | 23 |
| −3 | 24 | 24 |
| −4 | 25 | 25 |
| −5 | 26 | 26 |

The analysis template 600 can be used to track the statistical data generated during development/training of the ML model 104. For example, the processing system 102 can track the occurrences of certain mutations according to the sequence location 614 or the identifier for the corresponding entry 610 and the indel mutation offset/identifier. The processing system 102 can use the counted occurrences for each sample, each sample set, or a combination thereof to compute the correlation between the mutations and the onset of the corresponding type of cancer.

In some implementations, the processing system 102 can calculate the number of occurrences for each of the expected and/or derived phrases, such as for indel variants with or without indel variant ‘0,’ in the patient sequencing data. For each set of phrases associated with a particular indel variant type, the processing system 102 can calculate a statistical value (e.g., a median value) from the set of the number of occurrences. The median value can represent the counts associated with the particular TRS with a particular type of indel variant in the corresponding patient.

As an illustrative example, the processing system 102 can process three TR sequences derived from a targeted k=16 wild-type nucleotide (e.g., ATCATCATC) as shown below in Table 2.

TABLE 2

| TR Sequence Associate K-mers | K-mer |
|---|---|
| (Underlined) | Count |
| . . . ACTTGAATCATCATCATCCTCCTA . . . | 7 |
| (SEQ ID NO: 10) | |
| . . . ACTTGAATCATCATCATCCTCCTA . . . | 11 |
| (SEQ ID NO: 11) | |
| . . . ACTTGAATCATCATCATCCTCCTA . . . | 10 |
| (SEQ ID NO: 12) | |

The processing system 102 can calculate the median value of the counts as 10. Accordingly, the processing system 102 can assign a count of 10 to a corresponding TR sequence indel type (e.g., indel type +1) for this patient.

The analysis template 600 is shown for exemplary purposes as a template with a general layout for organizing information for each of the segments and/or phrases. It is understood that the analysis template 600 can include different categorizations and arrangements with additional or different pieces of information. Further, it is understood that an active or “in use” version of the genome TR reference catalogue 230 can be populated with values corresponding to the various categories of the entries 610.

In addition to carefully selecting the processing parameters (e.g., the indel variant value 512 and/or the segment length 420) and reducing the overlaps 352 in the unique segments 360 described above, the processing system 102 can further increase the processing efficiencies and accuracy of the ML model 104 by removing duplicate phrases or k-mers. The processing system 102 can inadvertently introduce or generate the duplicate phrases since the derived phrases 510 are generated by altering the unique segments 360. In other words, the derived phrases 510 may include character sequences that match other phrases corresponding to other portions of the human genome (e.g., derived and/or unique phrases corresponding to different locations or TR combinations). The processing system 102 can use the refinement mechanism 115 (e.g., the duplicate filter 254 (FIG. 2)) to identify and remove such duplicated phrases.

In some implementations, the duplicate filter 254 can be configured to compare the derived phrases 510 to the expected phrases 410 corresponding to different locations in the human genome. Additionally or alternatively, the duplicate filter 254 can be configured to compare the derived segments 560 to the unique segments 360 associated with other locations. Moreover, the duplicate filter 254 can compare the derived phrases 510 and/or derived segments 560 across different locations to find matches. For example, the processing system 102 can sort the phrases according to the unique segments 360 and/or the repeated base unit 356 and then according to the base unit length 424. The duplicate filter 254 can be configured to remove one or more or all of the instances of the matching phrases (having, e.g., same base TR units and TR-pattern length). In other words, the duplicate filter 254 can remove from further processing character combinations representative of sequences/mutations that can be found at multiple locations in the human genome. Accordingly, the processing system 102 can ignore the potentially misleading character patterns in analyzing for correlations to different types of cancers and reduce the overall number of processed phrases.

### Downstream Filtering

In addition to the text-based filtering described above, the processing system 102 can further filter the data and/or the processing results. For example, the processing system 102 can use the quality filter 256 (FIG. 2) to preprocess and/or adjust for the input patient data, such as the DNA sample set 206. The processing system 102 can use the quality filter 256 to reduce, remove, or adjust for imperfections (e.g., biases caused by inaccurate/insufficient reads) that may be introduced by sequencing technologies. In some implementations, the quality filter 256 can adjust for or normalize different read depths (e.g., the number of times that a given nucleotide in the genome was detected in a sample) across the separately sequence data, such as across the cancer-free data 210, the non-regional data 211, and/or the cancer-specific data 212.

To adjust for the different read depths, the quality filter 256 can be configured to require minimum read depths for the input patient data. In other words, the quality filter 256 can remove or filter out samples and/or corresponding sequenced strings having the sample read depth 214 (FIG. 2) less than a predetermined threshold (e.g., 10). Additionally or alternatively, the quality filter 256 can be configured to normalize the read depths to a predetermined depth (e.g., 200) across the different data sets. In normalizing the read depth, the quality filter 256 can calculate a scale factor for each data set by dividing the predetermined depth by the corresponding sample read depth 214. The scale factor can be applied or multiplied to wild-type counts (e.g., number of character sequences/segments corresponding to genes found in natural non-mutated form) for the set, thereby calculating the normalized wild-type count. Similarly, the quality filter 256 can apply the scale factor to the mutation counts (e.g., indel counts) found in each corresponding set. Accordingly, the wild-type counts and the mutations counts for the different data sets can be normalized to a common predetermined read depth using the scale factor.

Additionally or alternatively, the quality filter 256 can be configured to remove nucleotides having sub-standard quality. For example, the quality filter 256 can be configured to filter out data samples or strings having the sample quality score 216 (FIG. 2), such as the Phred quality score, below a predetermined quality threshold (e.g., 20). The quality filter 256 can replace characters for the substandard nucleotides to a predetermined character (e.g., ‘N’).

The processing system 102 can further use the comparison correction filter 258 (FIG. 2) to remove computational noise or errors. Even with the reduced number of computations, the number of computations and comparisons may inadvertently introduce false positives. Accordingly, the comparison correction filter 258 can be configured to correct the intermediate data, such as using a Bonferroni correction process. For example, the comparison correction filter 258 can adjust (by, e.g., dividing) a predetermined somatic classification threshold (p-value criteria, such as 0.01) by the number of phrases being processed/compared.

Moreover, the processing system 102 can use the fraction filter 260 (FIG. 2) to remove or adjust for physiological features and/or collection-based features that interfere with the data processing. In some implementations, the fraction filter 260 can be configured to address samples having relatively low numbers of derived phrases (e.g., sample sets having mutant counts less than a predetermined threshold). For example, the fraction filter 260 can include an allelic fraction filter. The allelic fraction for sample/data can be calculated based on dividing the number of derived phrases 510 by a sum of wild-type counts and mutant counts. The fraction filter 260 can classify data/strings as not being somatic when the corresponding allelic fraction values are less than a predetermined threshold (e.g., 0.05).

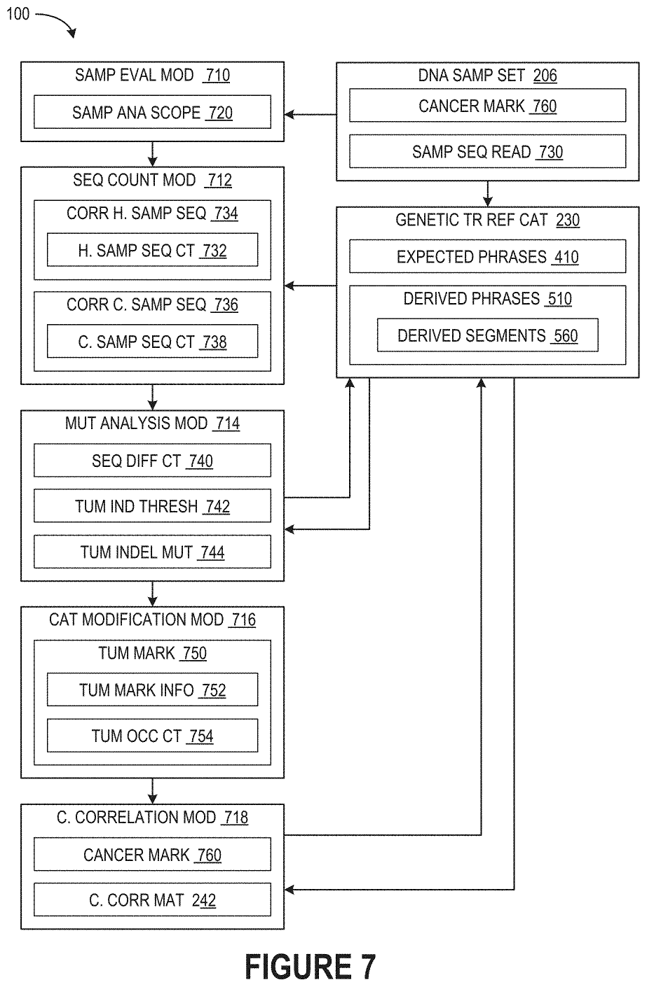

FIG. 7 shows a control flow diagram illustrating the functions of the computing system 100 in accordance with various implementations. The computing system 100 can be implemented to supplement and refine information in the genome TR reference catalogue 230 with information from the DNA sample sets 206 based on the unique segments 360 and the various phrases. In general, the computing system 100 can analyze one or more of the DNA sample sets 206 to process (1) mutations at specific locations of DNA sequences, (2) correlation of mutation patterns, (3) corresponding indications of one or more types of cancer, or a combination thereof. The functions of the computing system 100 can be implemented with a sample set evaluation module 710, a sequence count module 712, a mutation analysis module 714, a catalogue modification module 716, a cancer correlation module 718, or a combination thereof.

The evaluation module 710 can be configured to evaluate the scope of the DNA sample set 206, including the cancer-free data 210, the non-regional data 211, and/or the cancer-specific data 212. For example, the evaluation module 710 can evaluate the DNA sample set 206 to identify factors, properties, or characteristics thereof to facilitate analysis of the different categories of data. In some implementations, the evaluation module 710 can be optional. The evaluation module 710 can generate a sample analysis scope 720 for the DNA sample set 206. The sample analysis scope 720 is a set of one or more factors that may govern/control the analysis of the DNA sample set 206. For example, the sample analysis scope 720 can be generated based on the supplemental information 220. The sample analysis scope 720 can be used to identify usable phrases (e.g., the expected phrases 410 and/or the derived phrases 510) based on the sequence location 614 and the phrase length k 416.

The computing system 100 can receive the derived phrases 510 and associated information from the genome TR reference catalogue 230 and/or the DNA sample set 206. The mutation analysis mechanism can be implemented with the count module 712 and the analysis module 714. The count module 712 may be responsible for calculating a number of occurrences (e.g., a sequence count) for specific DNA sequences/phrases in a sample set. The count module 712 can calculate the sequence count based on a number of sample sequence reads 730, such as the sequence reads for the DNA fragments in one or more categories of data in the DNA sample set 206.

For the cancer-free data 210, the count module 712 can calculate a healthy sample sequence count 732 for each instance of a corresponding healthy sample sequence 734 identified in the cancer-free data 210. The corresponding healthy sample sequence 734 is a DNA sequence in the healthy sample DNA information 734 that corresponds to one of the derived segments 560 and/or the derived phrases 510. The heathy sample sequence count 732 is the number of times that the corresponding healthy sample sequence 734 is identified in the cancer-free data 210. Similarly, for the cancer-specific data 212 and/or the non-regional data 211, the count module 712 can calculate count values for each instance of a targeted sequence identified in the data group. In other words, the count module 712 can calculate the number of times the various phrases are found within the samples according to the corresponding categories.

The count module 712 can identify the corresponding healthy sample sequence 734 and the corresponding cancerous sample sequence 738 for a given expected phrase, and more specifically the derived phrase. For example, the sequence count module 712 can search through the different categories of data for matches to one or more of the derived segments within the corresponding phrases. As one specific example, the count module 712 can search for a string of consecutive base pairs that matches one of the derived segments 560 of the derived phrases 510.

The count module 712 can calculate the healthy sample sequence count 732 as the total number of each of the corresponding healthy sample sequence 734 identified in each of the sample sequence reads 730 in the cancer-free data 210. In many cases, the corresponding healthy sample sequence 734 will correspond with a single instance of the tandem repeat indel variants 310. In these cases, the total value of the healthy sample sequence count 732 will be equal to the total number of the sample sequence reads 730 in the cancer-free data 210. For example, where the cancer-free data 210 includes 50 instances of the sample sequence reads 730 per DNA segment, the healthy sample sequence count 732 for a given instance of the corresponding healthy sample sequence 734 should also be 50. The case of non-unity between the number of sequencing reads and the healthy sample sequence count 732 can generally be attributed to sequencing errors.

In many cases, the corresponding healthy sample sequence 734 will match with the phrase with the indel variant value 312 of zero (e.g., the expected phrase with no insertions or deletions of the unique segment 360). However, in some cases, the corresponding healthy sample sequence 734 can differ. The differences between the corresponding healthy sample sequence 734 and the phrase with the indel variant value 312 of zero can account for wild type variants (e.g., naturally occurring variations) in the cancer-free data 210.

Similarly, the count module 712 can calculate the cancerous sample sequence count 736 for each of the corresponding cancerous sample sequence 738 that appear in the sample sequence reads 730 in the cancer-specific data 212. Due to possible mutations, the cancer-specific data 212 can include multiple different instances of the corresponding cancerous sample sequence 738 matching different instances of the derived segments 560, with each corresponding cancerous sample sequence 738 having varying values of the cancerous sample sequence count 736. As an example, in some cases, the corresponding cancerous sample sequence 738 and cancerous sample sequence count 736 will match with the corresponding healthy sample sequence 734 and healthy sample sequence count 732, indicating no mutations. As another example, for a given instance of the derived phrase 510, the cancer-specific data 212 may have a split in the cancerous sample sequence count 736 between the cancerous sample sequence 738 that is the same as the corresponding healthy sample sequence 734 and one or more other instances of the indel variants. For a given instance of the derived phrase 510, the count module 712 can track the cancerous sample sequence count 736 for each different instance of the corresponding cancerous sample sequence 738 in the cancer-specific data 212.

The flow can continue to the analysis module 714. The analysis module 714 may be responsible for determining whether a mutation exists in the corresponding cancerous sample sequence 738 of the cancer-specific data 212. In general, the existence of a mutation in the cancer-specific data 212 can be determined based on differences in the repeated TR patterns between the corresponding heathy sample sequence 734 and the corresponding cancerous sample sequence 738. More specifically, a difference in the number of the repeated base unit 356 can represent the existence of an indel mutation (e.g., a mutation corresponding to an insertion or a deletion of the repeated TR unit), such as for cancer-specific data 212 in comparison to the cancer-free data 210. For example, the analysis module 714 can determine that a mutation exists when the corresponding cancerous sample sequence 738 matches one of the derived segments 560 and/or the derived phrases different than that of the corresponding healthy sample sequence 734. In another example, the analysis module 714 can determine the difference between the corresponding healthy sample sequence 734 and the corresponding cancerous sample sequence 738 based on a sequence different count 740 (e.g., the total number of corresponding cancerous sample sequences 738 differing from the corresponding healthy sample sequences 734). In the case where the sequence difference count 740 indicates no differences, such as when the sequence difference count 740 is zero, the analysis module 714 can determine that no mutation exists in the corresponding cancerous sample sequence 738.

In general, the analysis module 714 can determine that an indel mutation has occurred when the sequence difference count 740 is a non-zero value. In some implementations, the analysis module 714 determines whether the indel mutation is a tumorous indel mutation based on whether the sequence difference count 740 is greater than the error percentage of the approach or apparatus used to sequence the cancer-free data 210, cancer-specific data 212, or a combination thereof.

In another implementation, the analysis module 714 can determine whether the indel mutation is a tumorous indel mutation 744 based on a tumor indication threshold 742. The tumor indication threshold 742 is an indicator of whether the number of mutations for a particular sequence in the cancer-specific data 212 indicates the existence of a tumorous indel mutation 744. The tumorous indel mutation 744 may occur when the sequence difference count 740 exceeds a tumor indication threshold 742. As an example, the tumor indication threshold 742 can be based on a percentage between the total number of sample sequence reads 730 and the sequence difference count 740. As a specific example, the tumor indication threshold 742 can require a sequence different count 740 be greater than 70 percent of the sample sequence reads 730 for the cancer-specific data 212. In another specific example, the tumor indication threshold 742 can require the sequence difference count 740 be greater than 80 percent of the sample sequence reads 730 for the cancer-specific data 212. In another specific example, the tumor indication threshold 742 can require the sequence difference count 740 be greater than 90 percent of the sample sequence reads 730 for the cancer-specific data 212.

When the corresponding cancerous sample sequence 738 includes the tumorous indel mutation 744, the computing system 100 can implement the modification module 716 to update or modify the genome TR reference catalogue 230. Said another way, the computing system 100 can implement the modification module 716 responsive to determining that the corresponding cancerous sample sequence 738 includes the tumorous indel mutation 744. For example, the modification module 716 can modify the genome TR reference catalogue 230 by identifying the instance of the catalogue entries 610 as a tumor marker 750 when the tumorous indel mutation 744 exists in the corresponding cancerous sample sequence 738.

The catalogue entries 610 that are identified as a tumor marker 750 can be modified by the modification module 716 to include tumor marker information 752. Some examples of the tumor marker information 752 can include a tumor occurrence count 754, such as the number of times that the tumorous indel mutation 744 was identified in a particular instance of the segment/phrase (e.g., TR pattern) for a given form of cancer. As a specific example, the tumor occurrence count 754 can be compiled from analysis of the DNA sample sets 206 for numerous cancer patients.

In another example, the tumor marker identification 752 can include information about the different instances of the corresponding cancerous sample sequence 738 matching to different instances of the derived segments/phrases along with the cancerous sample sequence count 736, the total number of sample sequence reads 730 of the DNA sample set 206, all or portions of the supplemental information 220, or a combination thereof. In a further example, the tumor marker information 752 can include the number of repeated base units 356 in the corresponding cancerous sample sequence 738 that were different from the corresponding healthy sample sequence 734.

The tumor marker information 752 can include information based on the supplemental information 220. For example, the tumor marker information 752 can include the supplemental information 220 (e.g., source information), such as the cancer type, the stage of cancer development, organ or tissue from which the sample was extracted, or a combination thereof. In another example, the tumor marker information 752 can include the supplemental information 220 of the patient demographic information, such as the age, the gender, the ethnicity, the geographic location of where the patient resides or has been, the duration of time that the patient stayed or resided at the geographic location, predispositions for genetic disorders or cancer development, or a combination thereof.

The computing system 100 can use one or more instances of the segments/phrases identified as the tumor marker 750 to generate the cancer correlation matrix 242 with the correlation module 718. For example, the correlation module 718 can identify cancer markers 760 based on the tumor occurrence count 754 for each of the tumor markers 750 in the genome TR reference catalogue 230. The cancer markers 760 can correspond to mutation hotspots that are specific to indel mutations in instances of the TR patterns. In one implementation, the correlation module 718 can identify the cancer markers 760 based on regression analysis. For example, the regression analysis can be performed with a receiver operating characteristic curve to the optimum sensitivity and specificity from the tumor markers 750, tumor occurrence count 754, or a combination thereof to determine the cancer markers 760.

In another implementation, the correlation module 718 can identify the cancer markers 760 based on a ratio between, or percentage of, the tumor occurrence count 754 for the tumor marker 750 and the total number of the DNA sample sets 206 of a particular form of cancer that have been analyzed for the tumor marker 750. As a specific example, the correlation module 718 can identify the cancer markers 760 as the tumor markers 750 when the ratio between the tumor occurrence count 754 and the total number of DNA sample sets 206 that are analyzed is 90 percent or more of the DNA sample sets 206 for a particular form of cancer. In this case, the cancer correlation matrix 242 can include the cancer markers 760 that were identified in this manner.

In a further implementation, the correlation module 718 generates the cancer correlation matrix 242 as the tumor markers 750 that are common among a percentage of the DNA sample sets 206 for a particular form of cancer are found. For example, the correlation module 718 can generate the cancer correlation matrix 242 as the tumor markers 750 appear in 90 percent or more of the total number of DNA sample sets 206. In other implementations, the correlation module 718 can generate the cancer correlation matrix 242 through other methods, such as regression analysis or clustering.

The correlation module 718 can generate the cancer correlation matrix 242 taking into account the supplemental information 220, such as the patient demographic information, to generate the cancer correlation matrix 242 for sub-populations. For example, the correlation module 718 can generate the cancer correlation matrix 242 based on the patient demographic information specific to gender, nationality, geographic location, occupation, age, another characteristic, or a combination of characteristics.

The computing system 100 has been described in the context of modules that perform, serve, or support certain functions as an example. The computing system 100 can partition or order the modules differently. For example, the evaluation module 710 could be implemented on the processing system 102, while the count module 712, analysis module 714, and correlation module 718 could be implemented on another computing device (also called the “external computing device” or simply “external device”) separate from the computing system. Alternatively, the processing system 102 can include the various modules described above.

The computing system 100 can implement the refinement mechanism 115 (FIG. 1A) via one or more or different modules described above. For example, the computing system 100 can include/implement the quality filter 256 in the sample evaluation module 710. Also, the computing system 100 can include/implement the consecutive overlap filter 252 and/or the duplicate filter 254 in the count module 712 (e.g., before or in preparation for the counting operations described above). Moreover, the count module 712 and/or the analysis module 714 can include the comparison correction filter 258 and/or the fraction filter 260.

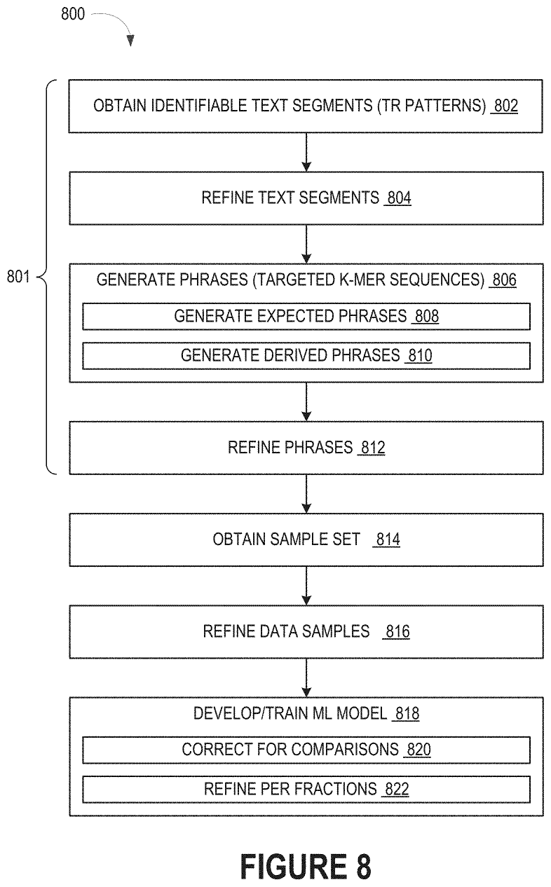

FIG. 8 shows a flow chart of a method 800 for processing and refining DNA-based text data for cancer analysis in accordance with one or more implementations of the present technology. The method 800 can be implemented using the computing system 100 (FIG. 1A) including the processing system 102 (FIG. 1A). The method 800 can be for developing the ML model 104 (FIG. 1A) including generating the various phrases and refining the processing results (via, e.g., the refinement mechanism 115 (FIG. 1)) as described above.

The method 800 includes the computing system 100 obtaining identifiable text sequences (e.g., TR-based patterns) at block 802. In some implementations, the processing system 102 can obtain the identifiable text sequences based on generating the unique segments 360 (FIG. 3) from the reference data 112 (FIG. 1A), such as by generating the character patterns representative of the identifiable TR patterns the human genome. In other implementations, the processing system 102 can access/receive the unique segments 360 generated by an external device.

The obtained unique segments 360 can serve as an initial set of segments representative of TR sequences. Each segment in the initial set can include N number of adjacently repeated base units 356. The repeated base units 356 for the initial set can have the base unit length 424 that is uniform across the segments.

At block 804, the computing system 100 can refine the identifiable text segments, such as by using/implementing the consecutive overlap filter 252 (FIG. 2). In some implementations, the processing system 102 can refine the identifiable text segments by removing the overlaps 352 (FIG. 3A), such as the TR patterns that are consecutive of and/or overlap each other, from the initial set of the unique segments 360 as described above. The processing system 102 can generate a refined set of the segments based on removing the overlaps 352 from the initial set.

At block 806, the computing system 100 can generate the phrases, such as the k-mer sequences targeted for use in subsequent data processing. For example, at block 808, the processing system 102 can generate the expected phrases 410 (FIG. 4). The processing system 102 can use the unique segments 360 (e.g., uniquely identifiable TR patterns) to generate the expected phrases 410, such as by adding different combinations of the flanking text 414 (FIG. 4) as described above. Also, at block 810, the processing system 102 can generate the derived phrases 510 (FIG. 5). The processing system 102 can use the expected phrases 410 to generate the derived phrases 510, such as by adjusting the unique segments 360 within the expected phrases to the derived segments 560 representative of indel mutations as described above.

In some implementations, the generated phrases can serve as an initial set. The generated phrases can correspond to different locations within the human genome. For example, the phrases can have the phrase length k 416 and include (1) location-specific TR-based segments (e.g., expected phrases 410) and/or (2) indel derivations of the TR-based segments adjacent to corresponding sets of flanking texts (e.g., derived phrases 510).

At block 812, the computing system 100 can refine the set of phrases, such as by using/implementing the duplicate filter 254 (FIG. 2). For example, the processing system 102 can refine the expected phrases 410 and/or derived phrases 510 by removing the duplicates or representations of DNA sequences or mutations that may correspond to more than one location. In other words, the processing system 102 can search for inadvertently generated representations of mutations that match mutations or expected/healthy sequences corresponding to a different location in the human genome as described above.

The operations described above for one or more of the blocks 802-812 can correspond to a block 801 for generating text phrases that represent different DNA sequences. The generated text phrases can represent various uniquely identifiable DNA sequences and mutations sequences for TR indel variants. The generated/refined text phrases can be used to determine correlations between the various mutations and onset cancer in the DNA sample set 206.

At block 814, the computing system 100 can obtain one or more sample sets (e.g., the DNA sample set 206 (FIG. 2)). In some implementations, the processing system 102 can receive sequenced DNA data from publicly available databases, healthcare providers, and/or submitting patients. The obtained data sample sets can include corresponding or known diagnoses, such as categorizations or tags identifying that the DNA data is from patients confirmed to be without cancer or confirmed to have specific cancers. Additionally, the obtained data can include physiological source locations of the DNA data. For samples sourced from the patients having cancer, the source locations can be the cancerous tumor or a location different from or unrelated to the malignant tumors. Accordingly, the processing system 102 can include a combination of the cancer-free data 210, the non-regional data 211, and the cancer-specific data 212, illustrated in FIG. 2. The obtained DNA sample set 112 can further include other details, such as the supplemental information 220 (FIG. 2), the sample read depth 214 (FIG. 2), the sample quality score 216 (FIG. 2), or the like.

At block 816, the computing system 100 can refine the data samples 816, such as by using/implementing the quality filter 256 (FIG. 2). For example, the processing system 102 can identify the characters corresponding to nucleotides having Phred scores less than the quality threshold. The processing system 102 can replace the identified characters with a predetermined dummy letter as described above. Additionally or alternatively, the processing system 102 can filter and/or adjust for nonuniform read counts or read depths across the DNA sample set 206. The processing system 102 can remove sample data having the sample read depth 214 below a depth requirement/threshold as described above. The processing system 102 can also adjust for the nonuniformity by calculating and applying the scale factor to the read counts as described above.

At block 818, the computing system 100 can develop and train the ML model 104 using the refined phrases and the refined data samples. For example, the processing system 102 can count and analyze the various somatic mutations, compute correlations between the mutations and cancers, and the like as described above. Using the results, the processing system 102 can select a set of features that include phrases having sufficient correlations to one or more types of cancers. The processing system 102 can design and train the ML model 104 using the selected features (e.g., correlative phrases representative of cancer-causing somatic mutations).

In developing and training the ML model 104, the processing system 102 can further refine the intermediate processing results. For example, at block 820, the processing system 102 can correct for comparison noises, such as by using/implementing the comparison correction filter 258 (FIG. 2). The processing system 102 can correct for the comparison noises using the p-value criteria as described above. Also, at block 822, the processing system 102 can refine the intermediate results per the fractional features. The processing system 102 can use the fraction filter 260 (FIG. 2) in classifying or distinguishing between somatic and non-somatic mutations.

The processing system 102 can develop/train the ML model 104 such that the model is configured to compute a cancer signal based on analyzing text-based patient DNA data according to represented somatic indel mutations in patient DNA. The processing system 102 can develop/train the ML model 104 based on computing correlations between mutations (as represented by the derived phrases) and onset/existence of one or more types of cancers as represented by the DNA sample set 206. Using the correlations, the ML model 104 can be configured to compute the cancer signal that represents (1) a likelihood that a corresponding patient has developed the one or more types of cancer, (2) a likelihood that the patient will develop the one or more types of cancer within a given duration, and/or (3) a development status at least leading up to onset of one or more types of cancer.

### Approaches to Selecting Features for Improved Cancer Detection

In one aspect, the present disclosure is directed toward AI and ML mechanisms that can be used to select features for detecting cancer through analysis of genetic information. For the purposes of illustration, implementations may be described in the context of a DNA sample set (e.g., DNA sample set 206) that includes genetic information in the form of DNA sequences that are associated with, or representative of, cancer-free data 210, non-regional data 211, and/or cancer-specific data 212. Said another way, the DNA sample set may include genetic information generated for a cancer-free sample, a sample taken from a non-cancerous region, or a cancerous sample.

At a high level, the approach described above involves obtaining data that includes (i) DNA sequences (e.g., in the form of cancer-free data 210 or non-regional data 211) corresponding to non-cancerous samples and (ii) DNA sequences (e.g., in the form of cancer-specific data 212) corresponding to cancerous samples. The former may be referred to as “non-cancerous DNA sequences” or “reference DNA sequences,” and the latter may be referred to as “cancerous DNA sequences.” Moreover, because this data is to be used in the training of the ML model 104, this data may be referred to as a “training dataset.” The training dataset can be processed by a computing system (e.g., computing system 100 of FIG. 1A)—and more specifically, a processing system (e.g., processing system 102 of FIG. 1A)—to identify an initial set of unique segments 360 (FIG. 3B) and corresponding segment locations 364 (FIG. 3B) that identify positions (e.g., first letter positions) of the segments within a target sequence 354 (FIG. 3B) as discussed above. Each unique segment 360 may be representative of a sequence of nucleotides that uniquely corresponds to a molecular position within the human genome.

The computing system 100 can process the training dataset according to unique locations or markers. For example, the computing system can generate a list of unique TR-based patterns and indel variants thereof based on an analysis of flanking sequences (e.g., by examining leading nucleotides and trailing nucleotides) using a “sliding window approach.” In particular, a “sliding window” that has a predetermined width (e.g., defined by phrase length k 416 of FIG. 4) may be used to isolate successive portions within an expected phrase 410 that is representative of a DNA sequence. As the computing system 100 shifts the bounds of the sliding window, the information contained within the sliding window can be compared to a reference pattern (e.g., human genome or portions thereof) to verify target conditions, such as uniqueness across the human genome. When the target conditions are verified, the computing system 100 can retain the information within the sliding window as uniquely identifiable TRs. The computing system 100 can further process the uniquely identifiable TRs to identify potential mutations (e.g., indels that add to or delete from the sequence of interest). The computing system 100 can process and retain a set of potential mutations that may be unique and/or indicative of certain types of cancer.

As part of training or implementing the ML model 104, a DNA sample set 206 that includes DNA data (e.g., representative of a set of sequenced DNA information) can be provided as input, for analysis in accordance with the uniquely identifiable TRs and/or indel variants thereof. In other words, the computing system 100 can use the uniquely identifiable TRs and/or indel variants thereof to analyze the DNA data included in the DNA sample set 206. As mentioned above, the DNA sample set 206 can include genetic information (e.g., text-based representations) derived or extracted from human bodies. Thus, the computing system 100 can develop, train, or implement the ML model 104 based on analyzing instances or patterns of the uniquely identifiable TRs and/or variants thereof in relation to certain types of cancers. The locations of detected deviations and/or the patterns of detected deviations within the DNA data of the DNA sample set 206 may be aggregated to identify an initial set of indicators configured to predict onset of cancer, identify a likely onset of the predicted type(s) of cancer, detect existence and/or absence of cancer, identify the existing type(s) of cancer, or a combination thereof.

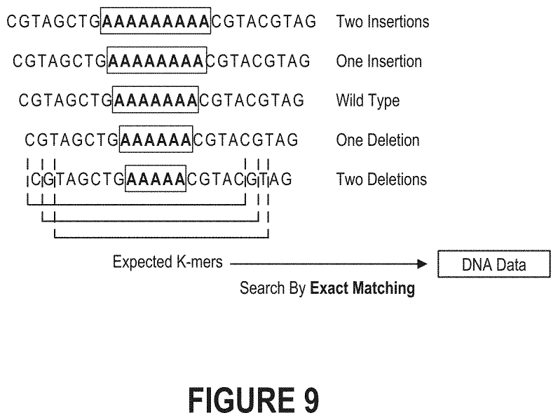

FIG. 9 illustrates how the computing system 100 can flexibly search for TR sequences with different indel mutations in expected phrases 410. As mentioned above, the expected phrases 410 may also be referred to as “k-mers.” At a high level, a TR sequence is a segment of a longer sequence that includes multiple repeated patterns that exceed a minimum number of base pairs. For example, each TR sequence can be selected based on the repeated base unit having the minimum number of base pairs ranging between five and eight base pairs.

In FIG. 9, the unique segment that is representative of the TR sequence has seven base pairs with a repeated base unit of one base pair ‘A.’ As such, an indel mutation of one deletion will result in a unique segment that has six base pairs with a repeated base unit of ‘A’ while an indel mutation of two deletions will result in a unique segment that has five base pairs with a repeated base unit of ‘A.’ similarly, an indel mutation of one insertion will result in a unique segment that has eight base pairs with a repeated base unit of ‘A’ while an indel mutation of two insertions will result in a unique segment that has nine base pairs with a repeated base unit of ‘A.’ It should be appreciated that these examples are shown solely for the purpose of illustration. Indel variants with more than two insertions or deletions could be part of the expected phrases 410.

Through the use of expected phrases 410 or “k-mers,” the computing system 100 can determine sequences of a given length (e.g., at least length n, where n is an integer greater than two) and then count the occurrences of the TR sequences and indel variants of interest. For example, the computing system 100 may parse reference data (e.g., reference data 112 of FIG. 1A) to discover the number of occurrences of a given TR sequence in sequencing reads corresponding to a non-cancerous sample (e.g., of tissue, bodily fluid, etc.).

Some challenges with mutation calling can be addressed by using the k-mers instead. First, mutation calling can be based on the human genome—which serves as a reference—rather than a patient-specific genome. Calculating all possible indel variants for a TR sequence across the human genome offers a flexible, reference-free approach to mutation calling. Second, the k-mers can be defined to cover sequences (e.g., corresponding to indel variants) that vary slightly from a TR sequence of interest as discussed above, allowing for more reliable mutation calling. This allows the computing system 100 to experience fewer errors in detecting TR sequences and indel variants thereof due to amplification issues, alignment issues, or the like. Simply put, relying on TR sequences and indel variants determined in the manner prescribed above lessens the likelihood of inaccuracy, for example, due to false positives or false negatives.

In samples taken from a human body, satellite DNA known as “msDNA” may be present. At a high level, msDNA is a complex of DNA, RNA, and possibly proteins that can be found in fluids like blood. msDNA can comprise a small, single-stranded DNA molecule that is linked to a small, single-stranded RNA molecule. One of the benefits of employing k-mers is that msDNA could be examined in addition to, or instead of, amplified DNA molecules. Through examination, the computing system 100 can identify the number of instances of each k-mer in a DNA sample set 206 regardless of its form. In particular, the computing system 100 can search the DNA sample set 206 by exact matching each k-mer against the DNA data included therein. At a high level, each target location included in the initial set of unique segments 360 can identify a molecular position.

As mentioned above, the mutations discovered by matching the k-mers against DNA data can be used to create, generate, or otherwise obtain target locations within the human genome. The DNA data could be associated with a single DNA sample set (and thus, a single patient), or the DNA data could be associated with multiple DNA sample sets (and thus, multiple patients). For example, the DNA data may be representative of genetic information corresponding to samples that were collected, characterized, and analyzed by a third party, such as a healthcare system or a research institution (e.g., The Cancer Genome Atlas), for a set of patients (e.g., several hundred or thousand patients). In such a scenario, each DNA sample set may be associated with the genetic information of a corresponding patient and a label that either indicates (i) the type of cancer with which the corresponding patient was diagnosed or (ii) that the patient was diagnosed as not having cancer. Through analysis of the DNA data, the computing system can establish a unique segment set 113 (FIG. 1A) as discussed above.

In some implementations, the computing system 100 uses a refinement mechanism 115 (FIG. 1A) to reduce the size of the unique segment set 113 to produce a refined set 116. For example, the computing system 100 may apply the refinement mechanism 115 to reduce the number of expected phrases 120 and derived phrases 122 that collectively correspond to the unique segment set 113, for example, by removing duplicate phrases and overlap phrases. By removing duplicate phrases and overlap phrases, the computing device 100 can avoid duplicative processing, namely, where the unique segment set 113 would indicate to look for instances of a given phrase at the same location or slightly different locations. By implementing the refined set 116 instead of the unique segment set 113, computational resources can be conserved (and issues such as duplicative processing, noise, and the like can be avoided). Further information regarding approaches to reducing the number of locations in the unique segment set 113 can be found in U.S. application Ser. No. 18/073,471, titled “Approaches to Reducing Dimensionality of Genetic Information Used for Machine Learning and Systems for Implementing the Same,” which is incorporated herein by reference in its entirety.

### Methodologies for Training and Implementing a Multiclass Model

Introduced here is an approach to training a multiclass model to classify a patient amongst multiple cancer types using sets of locations. These sets of locations may be part of a unique segment set 113 or a refined set 116 that are generated by a computing system (e.g., computing system 100 of FIG. 1A)—and more specifically, a processing system (e.g., processing system 102 of FIG. 1A)—in accordance with the approach described above. Assume, for example, that the processing system 102 receives input indicative of a request to train a multiclass model to classify patients among multiple cancer types based on an analysis of genetic information. Generally, the number of cancer types is based on the number of cancer types represented in the genetic information to be used as training data. For example, if the processing system 102 acquires the genetic information from TCGA as mentioned above, the multiclass model may be trained to classify patients among 32 cancer types. It will be understood that the multiclass model could be trained to classify patients among fewer than 32 cancer types or more than 32 cancer types. For example, it may be beneficial—from a resource consumption perspective—to limit training to fewer than 25, fewer than 20, fewer than 10 cancer types, or fewer than 5 cancer types. The cancer types for which the multiclass model is trained may correspond to the most common cancer types, or the cancer types for which the multiclass model is trained may correspond to similar physiological regions. As specific examples, a multiclass model could be trained to classify patients among different cancer types associated with the nose, throat, and lungs, or a multiclass model could be trained to classify patients among different cancer types associated with the immune system and blood-forming tissue such as bone marrow.

In response to receiving the input, the processing system 102 can obtain at least one set of locations for each cancer type of the multiple cancer types. As mentioned above, each set of locations may be representative of a unique segment set 113 or refined set 116. Accordingly, if the multiclass model is to be trained to classify patients among 32 cancer types, then the processing system 102 can obtain at least 32 sets of locations. The processing system 102 can then train the multiclass model using these cancer-specific sets of locations, so as to produce a trained multiclass model that is able to indicate the likelihood that a patient has any of the multiple cancer types upon being applied to corresponding genetic information. Thus, the trained multiclass model may produce likelihood values as output, and the number of likelihood values that are produced may correspond to the number of cancer types for which the multiclass model is trained.

The obtained set of locations can correspond to the unique segment set 113 generated in accordance with the sliding window described above. In some implementations, the locations in the unique segment set 113 may be further reduced to produce the refined set 116 as mentioned above, thereby improving the processing efficiency and/or lessening the required computational resources, such as by removing duplicates, predetermined patterns, or the like. Accordingly, the multiclass model could be trained using the unique segment set 113 or refined set 116 produced for each of multiple cancer types.

It has been found that the approach described below exhibits several notable advances, namely:
The ability to intelligently group, cluster, or otherwise combine the outputs (e.g., the likelihood values) produced by the multiclass model to gain insight into the health state of a patient through analysis of her genetic information. For instance, the outputs may surface biological insights related to metastatic patterns, cellular structure, physiological location, and the like. As an example, if the multiclass model outputs similar likelihood values for rectal cancer and colon cancer, then a targeted recommendation can be generated by the processing system 102. As another example, if the multiclass model outputs similar likelihood values for prostate cancer and brain cancer, then the processing system 102 may recommend testing for one cancer type (e.g., brain cancer) based on characteristics of the patient, ease of the testing process, etc. If testing for that cancer type does not reveal further results, then the healthcare professional responsible for performing or facilitating the testing may opt to test for the other cancer type (e.g., prostate cancer).
The ability to readily obtain proposed diagnoses for multiple cancers. As mentioned above, a multiclass model may produce a separate output (e.g., a likelihood value) for each type of cancer that the multiclass model is trained to detect. As such, the processing system 102 may be able to quickly gain insight into different cancer types (and more general categories, such as head and neck cancers). This can be particularly helpful if the multiclass model is trained to classify patients among multiple cancer types (e.g., more than 3, 10, 20, or 30 cancer types).
The ability to detect mutations that are indicative of a wide gamut of different cancer types allows for greater flexibility in testing. Since the multiclass model is not limited to a single cancer type, the multiclass model can be applied to genetic information acquired in different ways. For example, the multiclass model could be applied to genetic information that corresponds to sequencing reads of a tissue sample obtained from a potential tumor. As another example, the multiclass model could be applied to genetic information that corresponds to sequencing reads of a fluid sample acquired via liquid biopsy. Simply put, the breadth of the multiclass model allows for greater flexibility with respect to the origin of the genetic information to which the multiclass model is to be applied.

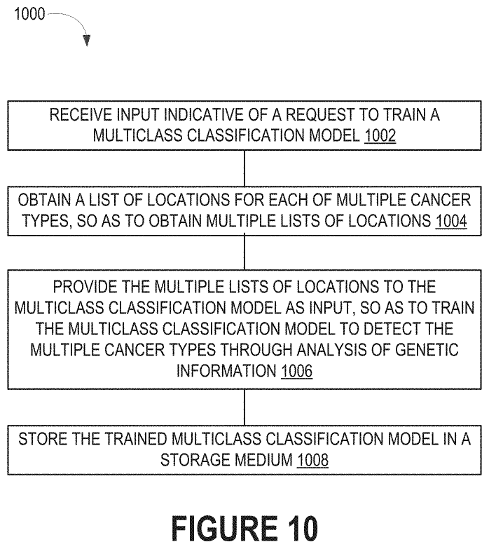

FIG. 10 includes a flow chart of a method 1000 for training a multiclass model to stratify patients among multiple cancer types based on an analysis of genetic information. For the purpose of illustration, the method 1000 is described as being performed by the processing system 102 (FIG. 1A). At block 1002, the processing system 102 can receive input indicative of a request to train the multiclass model. Generally, this input is provided through an interface that is generated by the processing system 102. Through the interface, an individual (also referred to as an “operator” or “administrator”) may select multiple cancer types for which the multiclass model is to be trained to detect. As an example, the individual may select all 32 cancer types for which genetic information is available from TCGA. As another example, the individual may indirectly select lists of locations associated with different cancer types as further discussed below, and the processing system 102 may identify the multiple cancer types based on the selected lists of locations.

At block 1004, the processing system 102 can obtain a list of locations for each of the multiple cancer types, so as to obtain multiple lists of locations. For example, the processing system 102 may employ a sliding window approach to create, based on comparisons of genetic information (e.g., included in, or derived from, a data sample set 206) to a reference human genome, a list of unique TRs that may be representative of mutations. This list of unique TRs may be referred to as the unique segment set 113. The process for obtaining unique segment sets is discussed in greater detail above. Note that, in some implementations, the processing system 102 may reduce unique segment sets by filtering some of the locations, thereby producing smaller lists of unique TRs. These smaller lists of unique TRs may be referred to as refined sets. The list of locations obtained for each cancer type may be representative of a unique segment set 113 or refined set 116.

For a given cancer type, the list of locations could be associated with a single sample (e.g., corresponding to a single patient) or multiple samples (e.g., corresponding to multiple patients). Thus, the list of locations obtained for each cancer type may be one of multiple lists of locations obtained for that cancer type. Generally, more than one sample is desired to ensure sufficient diversity in the underlying data to avoid overfitting of the multiclass model. Having multiple samples may also be important from a biological perspective. As an example, the processing system 102 may obtain genetic information for samples (and thus patients) that correspond to different stages of a given cancer type, so as to allow the multiclass model to learn how to distinguish between these different stages. As another example, the processing system 102 may obtain patient demographic information that can be included in the training data, so as to allow the multiclass model to learn how different characteristics are related to diagnostic outcome. Examples of patient demographic information include age, ethnicity, presence and prevalence (e.g., concentration) of biomarkers, family history of cancer, lifestyle habits (e.g., smoking), and the like. This information may be extracted from the medical record of the patient, or this information may be provided by the patient (e.g., through an interface generated by the processing system 102).

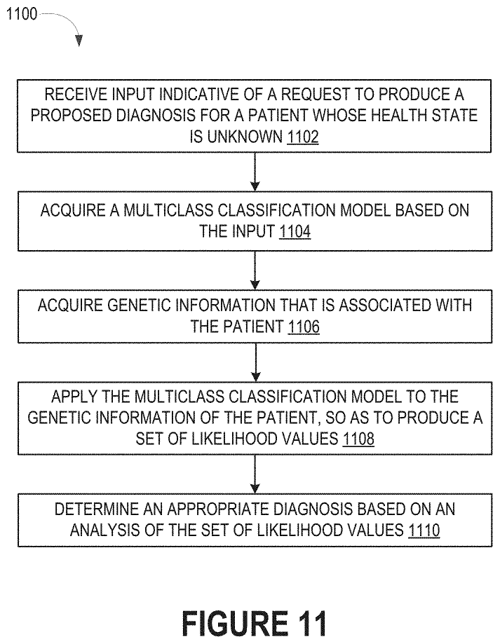

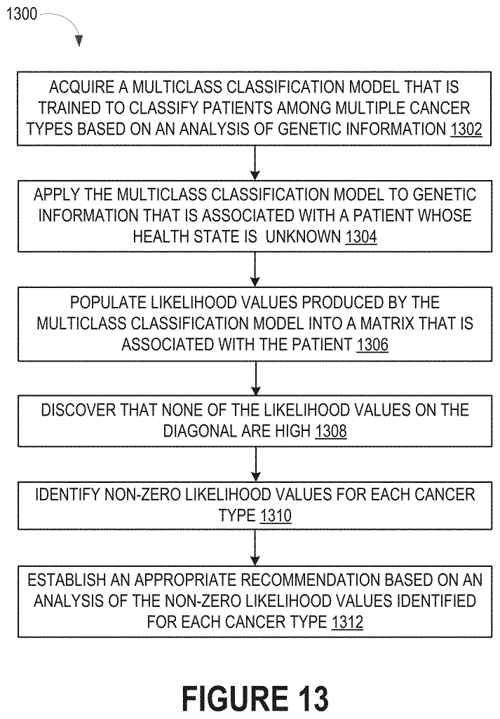

At block 1006, the processing system 102 can provide the multiple lists of locations to an untrained classification model as input, so as to produce a trained multiclass classification model. As discussed below with reference to FIGS. 11 and 13, upon being applied to genetic information associated with a patient whose health state is unknown, the trained multiclass model may produce, as output, a set of likelihood values that can be populated into a matrix. The set of likelihood values may include multiple series of values, each of which corresponds to a different cancer type. At block 1008, the processing system 102 can then store the trained multiclass model in a storage medium. As part of this process, the processing system 102 may associate contextual information with the trained multiclass model. For example, the processing system 102 may specify the multiple cancer types in metadata that is appended to the trained multiclass model. As another example, the processing system 102 may describe the source (e.g., TCGA) of the genetic information used as training data in metadata that is appended to the trained multiclass model. At a high level, the contextual information may be used by the processing system 102 to determine the scenarios where application of the trained multiclass model is appropriate, as well as identify when retraining is necessary (e.g., where new genetic information is available from the source).

FIG. 11 includes a flow chart of a method 1100 for applying a multiclass model that has been trained to stratify patients among multiple cancer types based on an analysis of genetic information associated with those patients. The multiclass model may be trained in accordance with the method 1000 of FIG. 10. Once again, the method 1100 is described as being performed by the processing system 102 (FIG. 1A) for the purpose of illustration.

At block 1102, the processing system 102 can receive input indicative of a request to produce a proposed diagnosis for a patient whose health state is unknown. Generally, this input is provided through an interface that is generated by the processing system 102. Through the interface, an individual (also referred to as an “operator” or “administrator”) may select or upload genetic information associated with the patient, either directly or indirectly. For example, the individual may identify the patient (e.g., via selection of a corresponding digital profile maintained for the patient), and the processing system 102 can then obtain the genetic information. As another example, the individual may select the genetic information itself, for example, by selecting the data structure in which the genetic information is stored. In some implementations, the individual may also select the cancer types for which diagnoses are desired. Alternatively, the processing system 102 may presume that the individual is interested in diagnoses for a wide range of cancer types (e.g., all 32 cancer types for which genetic information is available from TCGA).

In some implementations, the input can correspond to a preceding determination that the patient may be unhealthy or may have cancer as further discussed below. For example, upon receiving genetic information that is associated with the patient, the processing system 102 may apply a binary classification model thereto in order to produce an output. The binary classification model may be trained to indicate whether the patient is normal or not normal (and thus possibly suffering from cancer), or the binary classification model may be trained to indicate whether the patient has cancer or does not have cancer. The processing system 102 may perform the method 1100 only in response to a determination, based on the output produced by the binary classification model, that the patient is not normal or has cancer.

At block 1104, the processing system 102 can then acquire the multiclass model based on the input. In some implementations, the processing system 102 only maintains a single multiclass model (e.g., trained to detect at least two cancer types, 10 cancer types, 20 cancer types, 32 cancer types, or any other number of cancer types), and therefore the processing system 102 may simply acquire the multiclass model from a storage medium in response to receiving the input. In other implementations, the processing system 102 may maintain multiple multiclass models in the storage medium. For example, the processing system 102 may maintain a first multiclass model that has been trained to detect a first set of cancer types, a second multiclass model that has been trained to detect a second set of cancer types, etc. The different sets of cancer types which may correspond to different combinations or numbers of cancer types. The multiclass model may be selected from among the multiple multiclass models based on the input.

At step 1106, the processing system 102 can acquire genetic information that is associated with the patient. As mentioned above, the genetic information could be uploaded through the interface such that it is included in the input. Alternatively, the processing system 102 may acquire the genetic information from a source. The source could be internal to the computing system 100 of which the processing system 102 is a part (e.g., included in memory of the computing system 100), or the source could be external to the computing system 100. For example, the processing system 102 may obtain the genetic information from another computing device (e.g., a sequencing device or computer server). As a specific example, the processing system 102 could retrieve the genetic information from the medical record of the patient that has been made available (e.g., by the healthcare entity that manages the medical record or the patient herself).

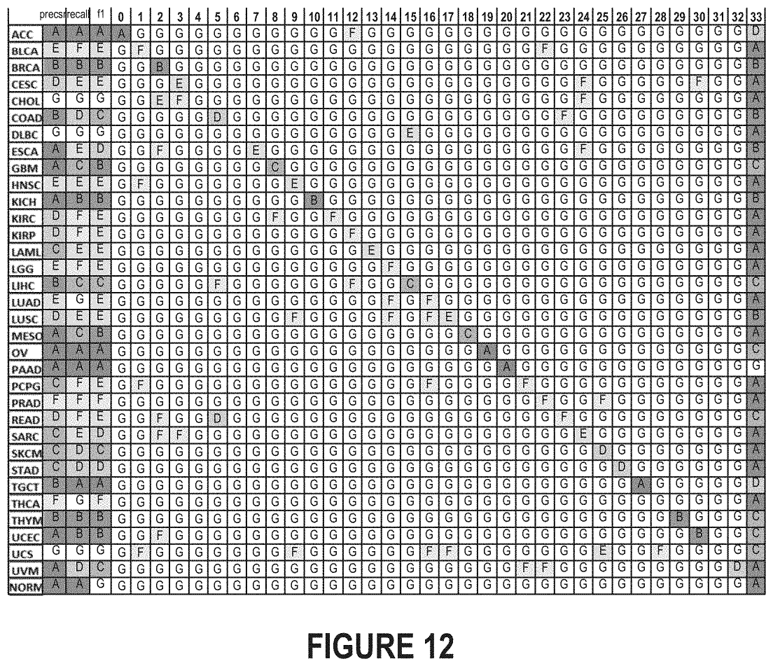

At block 1108, the processing system 102 can apply the multiclass model to the genetic information of the patient, so as to produce a set of likelihood values. The set of likelihood values may include multiple series of values, each of which corresponds to a different cancer type. As shown in FIG. 12, the set of likelihood values may be populated into a data structure, such as a matrix, for analysis purposes. At block 1110, the processing system 102 can then determine an appropriate diagnosis based on an analysis of the set of likelihood values. As discussed above, the processing system 102 may affirmatively predict a diagnosis for a given cancer type if the likelihood value on the diagonal is high. If none of the likelihood values on the diagonal are high—indicating that there is not a strong signal for any of the multiple cancer types—then the processing system 102 may analyze the other non-zero likelihood values included in each series as further discussed below with reference to FIG. 13. Accordingly, the processing system 102 may examine the set of likelihood values encoded in the matrix to determine a recommendation for treating a given cancer type or for establishing next steps for further diagnostic testing (e.g., in response to determining that multiple cancer types are predicted with similar likelihood).

FIG. 12 includes a chart illustrating a matrix of likelihood values output by a multiclass model upon being applied to genetic information associated with cancerous samples taken from patients known to have cancer. Specifically, the genetic information was obtained from TCGA, and therefore the health states of those patients were known. Said another way, it was known which cancer type was assigned to each sampled patient. Applying the multiclass model to genetic information associated with a sample taken from a patient whose health state is unknown may result in production of a matrix of comparable form (without the precision, recall, and F1 scores though, as the actual diagnosis is unknown).

In reviewing FIG. 12, there are several items worth mentioning. First, precision, recall, and F1 scores or ratings were produced for each cancer type. Second, the likelihood entries along the diagonal indicate the relative strength of the multiclass model to classify the corresponding cancer type. Ideally, the precision and recall results should be high, with the highest result (e.g., likelihood values or ratings) existing on the diagonal. When the highest likelihood value exists on the diagonal, it can be inferred that predictions of the corresponding cancer type are likely to be accurate. This relationship is generally proportional. As such, the higher the result along the diagonal, the higher the likelihood that predictions for the corresponding cancer type will be accurate. FIG. 12 illustrates the results using letter ratings (e.g., sequentially A, B, C, D, and F with A being the highest or most optimal result). In some implementations, the letter ratings can correspond to a predetermined range of likelihood values (e.g., A for likelihood values greater than 0.8, B for values between 0.6 and 0.8, etc.). Moreover, indicators could be used in combination with the letter ratings to indicate where each likelihood value values within the predetermined range. Referring again to the aforementioned example where A is used for likelihood values greater than 0.8, A+ could be used for likelihood values greater than 0.95, A could be used for likelihood values between 0.85 and 0.95, and A− could be used for likelihood values between 0.80 and 0.85. Other schemes could also be used. For example, the matrix may be populated with terms such as “none,” “low,” “moderate,” and “high” to indicate how strongly the likelihood values indicate the presence of the cancer types. In other implementations, the matrix can include the likelihood values computed by the multiclass model. The likelihood values included in each row of the matrix can sum to one.

However, there may also be other non-zero entries that may be interesting as further discussed below. In addition to a satisfactory result (e.g., a calculated number, such as a likelihood value, exceeding a predetermined threshold or falling within a predetermined range) on the diagonal, the multiclass model should also produce satisfactory results for precision. At a high level, precision indicates how strongly the processing system 102 is testing for “true positive” and “false positive.” Similarly, the multiclass model should produce satisfactory results for recall. At a high level, recall indicates how strongly the processing system 102 is testing for “true negative” and “false negative.” When (i) the highest likelihood value exists on the diagonal and (ii) precision and recall are high, it can be inferred that the genetic information provided to the multiclass model as training data is showing a “strong signal” of the corresponding cancer type (and thus, is supported by the various metrics).

Determining whether precision and recall are sufficiently “high” is an important aspect of establishing whether the multiclass model is being properly trained. The determination of whether the value is sufficient may not be static, but instead could be dynamically determined. Accordingly, for precision and recall, a value may be considered “high” if it exceeds a threshold that is representative of a static value per cancer type that can be adjusted based on factors such as cancer type, relationship to other cancers, metastatic nature of a patient's cancer, medical records, and other biomarkers (e.g., blood level of Prostate-Specific Antigen (PSA) for prostate cancer). Additionally or alternatively, the value may be compared to the signal from the matrix and the likelihood value on the diagonal.

Determining whether the likelihood value on the diagonal is “high” is an important aspect of establishing whether the multiclass model is likely to produce useful outputs (e.g., predictions regarding different cancer types). Generally, the focus is not simply on the absolute magnitude of the likelihood value on the diagonal, but the fact that a “row” will add up to one, so the higher the likelihood value on the diagonal, the stronger the signal is for the corresponding cancer type. Again, the likelihood value should be examined in the context of the metrics mentioned above. Note that other non-zero values may be instructive in some instances, especially when the likelihood value on the diagonal is not particularly strong (e.g., less than 0.5). In particular, these other non-zero values may provide insights through comparison to one another and the precision and recall values.

Whether any of the likelihood values are deemed “strong signals” may depend on the threshold imposed by the processing system 102. For example, the processing system 102 may determine that if none of the likelihood values produced by the multiclass model as output exceed a threshold, then those likelihood values may not indicate the presence of any of the cancer types for which the multiclass model was trained. Each value produced by the multiclass model as output can fall within a range defined by an upper bound and a lower bound. Generally, this range is 0-1, though this range could be 0-10, 0-100, or any other range. In some implementations, the threshold value is representative of the midpoint between the upper and lower bounds. In other implementations, the threshold value is higher than the midpoint (e.g., 0.6 or 0.7 for a range of 0-1) or lower than the midpoint (e.g., 0.3 or 0.4 for a range of 0-1).

There may be some cancer types where the precision and recall numbers are low and the highest likelihood value is not on the diagonal (or the likelihood value on the diagonal is not significantly greater than at least one other likelihood value). In such a scenario, it can be inferred that predictions of that cancer type will not be as clear based on the relative weakness of the likelihood value on the diagonal. The likelihood value on the diagonal may be considered “weak” if (i) the highest likelihood value is not located on the diagonal, (ii) there is not a clear highest likelihood value in the row, or (iii) even if the highest likelihood value is on the diagonal, the difference between the highest likelihood value and the next highest likelihood value is small (e.g., less than 0.1 or 0.2). Predictions for these cancer types are not as clear as those predictions produced for cancer types for which the highest likelihood value is on the diagonal. While the predictions may not be clear, the processing system 102 could still look at the other non-zero values along the same row for further information to continue additional analysis. It is worth noting that when the highest likelihood value is not on the diagonal, the precision and recall values are also likely to be low (e.g., below 0.5 or 50 percent).

When this occurs, the processing system 102 can further investigate why the genetic information provided to the multiclass model as input is not showing a “strong signal” for a given cancer type (and thus, is not supported as evidenced by the low values for precision and recall). Once again, the determination of whether a value for precision or recall is “low” may not be static, but instead could be dynamically determined. Accordingly, for precision and recall, a value may be considered “low” if it does not exceed a threshold that is representative of a static value per cancer type that can be adjusted based on factors such as cancer type, relationship to other cancers, metastatic nature of a patient's cancer, medical records, and other biomarkers (e.g., blood level of PSA for prostate cancer). Additionally or alternatively, the value may be compared to the signal from the matrix and the likelihood value on the diagonal.

To determine whether the likelihood value on the diagonal is “low,” the processing system 102 may not simply examine the absolute magnitude of the likelihood value on the diagonal. Because a “row” will add up to one, the higher the likelihood value on the diagonal, the stronger the signal is for the corresponding cancer type, though the determination of whether the likelihood value is “low” may still be factor based. Again, the likelihood value should be examined in the context of the metrics mentioned above

Note that the terms “low” and “high” refer to numeric value or a corresponding rating, rather than the informative value of a likelihood value or a metric value (e.g., for precision or recall). Even if a likelihood value is “low,” significant insight into health can be gained through analysis of the low likelihood value in the context of other non-zero likelihood values.

FIG. 13 includes a flow chart of a method 1300 for grouping together different cancer types based on the likelihood values produced by a multiclass classification model as output. At block 1302, a processing system 102 can acquire, from a storage medium, a multiclass model that is trained to classify patients among multiple cancer types based on an analysis of genetic information. Generally, this is done in response to receiving input indicative of a request to generate a proposed diagnosis for a patient whose health state is unknown. As mentioned above, this input could be provided through an interface generated by the processing system 102, for example, via selection of the patient or genetic information that is associated with the patient. Alternatively, the input may simply be representative of receipt of genetic information associated with the patient. In some implementations, the processing system 102 may infer that receipt of genetic information is representative of a request to analyze that genetic information. At block 1304, the processing system 102 can apply the multiclass model to genetic information that is associated with the patient. As discussed above, the genetic information may be representative of sequencing reads of a sample taken from the patient.

For each cancer type, the multiclass model may produce a series of values that indicate the likelihood of the patient having that type of cancer. Accordingly, the multiclass model may produce a set of likelihood values that includes multiple series of values, each of which corresponds to a different cancer type. At bock 1306, the processing system 102 can populate the set of likelihood values into a matrix that is associated with the patient, as shown in FIG. 12.

Insights into the health state of the patient can be gained through analysis of the matrix. For example, if the likelihood value on the diagonal for a given cancer type is high (e.g., above 0.7 or 0.8), then the processing system 102 may infer that there is a strong likelihood of the patient having the given cancer type. However, the processing system 102 may discover that none of the likelihood values on the diagonal are high, as shown at block 1308, in some instances. When the likelihood values on the diagonal are low, the processing system 102 may look at other signals or metrics for guidance. Additionally or alternatively, the processing system 102 may examine the non-zero likelihood values as indicators of where to look further. This can be done on a per-sample basis (e.g., for the entire matrix) or a per-cancer-type basis (e.g., for each row in the matrix).

In the event that the processing system 102 discovers none of the likelihood values on the diagonal are high, the processing system 102 may identify the non-zero likelihood values for each cancer type as shown at block 1310. For example, the processing system 102 may employ programmed heuristics to identify non-zero likelihood values of interest (e.g., within a certain range, such as 0.5-0.7 or 0.3-0.7) and then group these non-zero likelihood values of interest. As another example, the processing system 102 may apply a clustering algorithm to the non-zero likelihood values included in the matrix. The clustering algorithm may be designed, programmed, and trained to group comparable non-zero likelihood values together. These groups may be formed using predetermined threshold values or predetermined ranges of values, or these groups may be formed more dynamically based on where gaps between the non-zero likelihood values occur.

At block 1312, the processing system 102 can establish, infer, or otherwise determine an appropriate recommendation based on an analysis of the non-zero likelihood values identified for each cancer type. The recommendation may be based on the nature of the cancer types for which the multiclass model output non-zero likelihood values. As an example, if similar likelihood values are output for rectal cancer and colon cancer, then a targeted recommendation to test for those cancer types can be generated by the processing system 102. As another example, if similar likelihood values are output for prostate cancer and brain cancer, then the processing system 102 may recommend testing for a biomarker (e.g., blood level of PSA) to establish which of those cancer types is more likely. If testing for one of those cancer types (e.g., brain cancer) does not result in an affirmative diagnosis, then a healthcare professional can simply proceed with testing the other cancer type (e.g., prostate cancer).

The grouping or clustering of cancer types based on likelihood values output by the multiclass model can serve an important informative purpose. These groups or clusters may indicate which cancer types are comparable from a biological perspective—at least in terms of the locations of mutations. Moreover, these groups or clusters can help surface insights into cancer types that are difficult to detect. As an example, pancreatic cancer and kidney cancer have historically been difficult to detect since there are few symptoms in the early stages of the disease. However, if the multiclass model outputs a non-zero value for these cancer types, then the processing system 102 may recommend additional testing to more definitely confirm the presence or absence of these cancer types. In some implementations, this is done only if the likelihood values output by the multiclass model for the other cancer types on the diagonal are low. In other implementations, this is done whenever the likelihood values for these more difficult cancer types exceed a threshold (e.g., 0.1 or 10 percent, 0.2 or 20 percent, etc.).

### Multitier Classification of Cancer Presence and Type

As discussed above, the multiclass model can be designed and then trained to simultaneously test for multiple cancer types through analysis of genetic information. This allows the multiclass model to serve as a valuable tool for stratifying patients amongst different cancer types. From a diagnostic perspective, the multiclass model tends to be more useful as the number of cancer types among which it can stratify patients increases. Simply put, a multiclass model that is able to stratify patients among 5, 10, 20, or 30 cancer types may be more useful to healthcare professionals than a multiclass model that is able to stratify patients among 1, 2, or 3 cancer types. However, as the number of cancer types increases, so too does the amount of computational resources that are required by the processing system 102 to design, train, and implement the multiclass model (and the time needed to design, train, and implement the multiclass model). This can become problematic if the multiclass model is to be applied to the genetic information of tens, hundreds, or thousands of different patients, either sequentially or simultaneously.

Introduced here, therefore, is an approach in which diagnoses are predicted in an improved manner through the application of different models in “tiers” or “stages.” The approach may involve applying a model set to the genetic information of an individual in order to ascertain the health of the individual. The model set may include (i) a first model that is designed and trained to produce an output that indicates whether the individual is healthy, (ii) a second model that is designed and trained to produce an output that indicates whether the individual has cancer, or (iii) a third model that is designed and trained to produce multiple outputs, each of which indicates whether the individual has a corresponding cancer type of multiple cancer types. Generally, the first and second models are binary classification models while the third model is the multiclass model discussed above.

The model set could include different combinations of these models, as well as other models not described herein. For example, the model set could include the first and third models that are applied in sequence, such that the third model is applied only if the output produced by the first model indicates that the individual is not healthy. As another example, the model set could include the second and third models that are applied in sequence, such that the third model is applied only if the output produced by the second model indicates that the individual has cancer. As another example, the model set could include the first, second, and third models. In implementations where the model set includes all three models, the second model may only be applied if the output produced by the first model indicates that the individual is not healthy, and the third model may only be applied if the output produced by the second model indicates that the individual has cancer.

Note that, in some implementations, aspects of the first, second, and third models may be incorporated into a single “superset” model that when applied to genetic information corresponding to an individual, acts in a manner comparable to aforementioned model set. At a high level, the superset model may be representative of a multiclass model that produces outputs indicative of proposed classifications for different sets of classes. As an example, the superset model may produce a first output that indicates whether the individual is healthy or not healthy, a second output that indicates whether the individual has cancer or no cancer, and a third output that indicates which cancer types, if any, are most likely. The third output may include a series of values, each of which indicates the likelihood that the individual has a corresponding cancer type. The superset model can derive the multiple outputs via a simultaneous/combined process (e.g., using a comprehensive neural network that outputs the multiple outputs).

For the purpose of illustration, implementations may be described in the context of a model set that includes at least two models. However, aspects of those implementations may be similarly applicable if the processing system 102 applies a superset model rather than the model set.

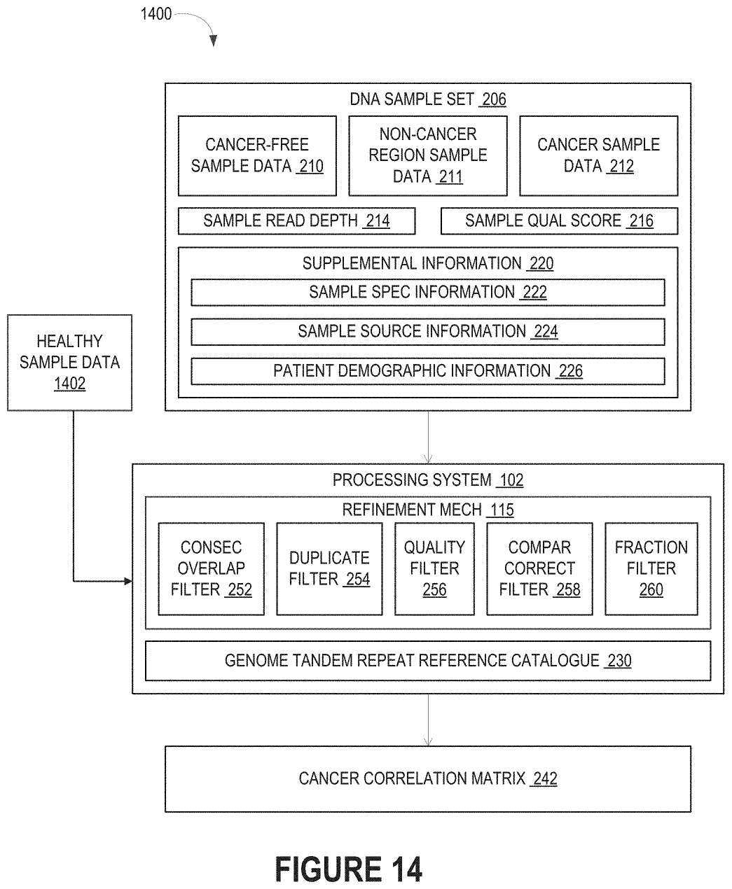

FIG. 14 includes another example data processing format for the processing system 102 in accordance with one or more implementations of the present technology. Specifically, FIG. 14 illustrates how the data processing format may be generally comparable to that of FIG. 2. Here, however, the processing system 102 obtains healthy sample data 1402 in addition to the cancer-free sample data 210, non-cancer region sample data 211, and cancer sample data 212. The non-cancer region sample data 211 and cancer sample data 212 for a particular instance of the DNA sample set 206 can correspond to samples taken from a single patient. For example, the cancer sample data 212 may correspond to sequenced DNA derived from a cancerous sample (e.g., a biopsy of a tumor) taken from the patient, while the non-cancer region sample data 211 may correspond to sequenced DNA derived from a non-cancerous sample (e.g., a biopsy taken from fluid or tissue other than the tumor) taken from the patient. Meanwhile, the healthy sample data 1402 may correspond to sequenced DNA derived from a sample taken from a healthy individual who shows no signs of having cancer. 210

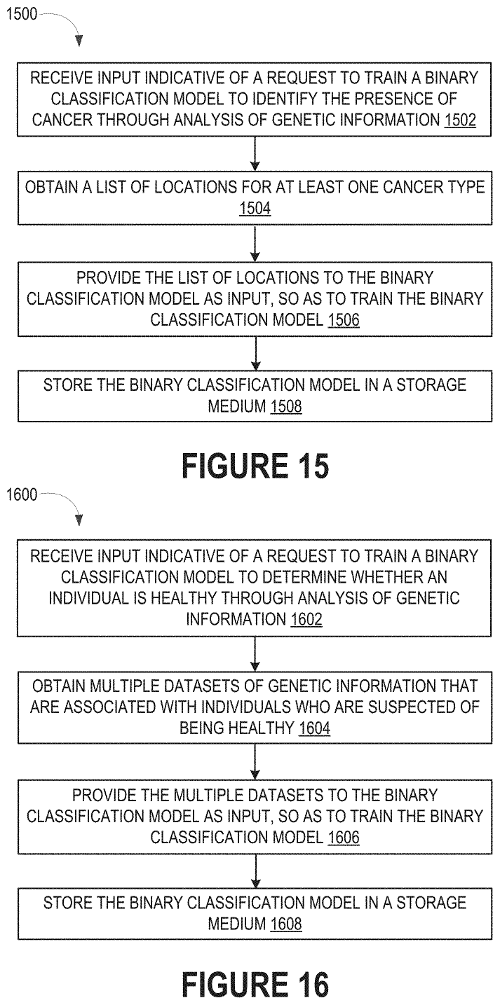

As discussed above with reference to FIG. 10, DNA sample sets 206 corresponding to a set of patients known to have different types of cancer may be used to train a multiclass model. In addition to the multiclass model, the processing system 102 may use the DNA sample sets 206 (and, more specifically, the lists of locations derived from the DNA sample sets 206) to train a binary classification model to identify the presence of cancer as further discussed below with reference to FIG. 15.

As shown in FIG. 14, the processing system 102 may also obtain, as input, healthy sample data 1402 that is associated with a healthy individual. The healthy sample data 1402 may be used by the processing system 102 to train another binary classification model to identify whether an individual is healthy based on an analysis of corresponding genetic information. Generally, the healthy sample data 1402 is representative of one of multiple datasets that are acquired by the processing system 102 for the purpose of training the other binary classification model. For example, the processing system 102 could acquire heathy sample data 1402 for tens, hundreds, or thousands of healthy individuals who show no signs of having cancer. At a high level, content of the healthy sample data 1402 can be similar to content of the cancer-free sample data 210, in that the underlying genetic information is associated with individuals who are not suspected of having cancer. However, the healthy sample data 1402 may be obtained via a different source than the cancer-free sample data 210. For example, the cancer-free sample data 210, non-cancer region sample data 211, and cancer sample data 212 may be obtained via one channel or from one source, while the healthy sample data 1402 may be obtained via another channel or from another source.

FIG. 15 includes a flow chart of a method 1500 for training a binary classification model to identify the presence of cancer based on an analysis of genetic information. For the purpose of illustration, the method 1500 is described as being performed by the processing system 102 (FIG. 1A). At block 1502, the processing system 102 can receive input indicative of a request to train the binary classification model. Generally, this input is provided through an interface that is generated by the processing system 102. Through the interface, an individual (also referred to as an “operator” or “administrator”) may indicate that the binary classification model is to be trained. Moreover, the individual may indicate the cancer types for which genetic information is to be used to train the binary classification model. As an example, the individual may select all 32 cancer types for which genetic information is available from TCGA, or the individual may select those cancer types for which at least a certain amount of genetic information (e.g., at least 5, 50, or 500 instances of cancer sample data 212) is available from a source. The source could be a network-accessible database, for example, managed by a healthcare system or research institution (e.g., TCGA).

At block 1504, the processing system 102 obtain a list of locations for at least one cancer type. Block 1504 of FIG. 15 may be comparable to block 1004 of FIG. 10, so long as the binary classification model is to be trained using locations associated with more than one cancer type. Lists of locations are normally obtained for a variety of different cancer types. Assume, for example, that the individual selects all 32 cancer types for which genetic information is available from TCGA through an interface generated by the processing system 102. In such a scenario, the processing system 102 can obtain a list of locations for each cancer type, so as to obtain multiple lists of target locations. Thus, the number of lists of locations that are acquired by the processing system 102 may match or exceed the number of cancer types to be included in the analysis performed by the binary classification model.

At block 1506, the processing system 102 can provide the list of locations to an untrained binary classification model as input, so as to produce a trained binary classification model. As mentioned above, the list of locations is normally one of multiple lists of locations if the untrained binary classification model is to be trained to detect mutations that are indicative of multiple cancer types. Upon being applied to genetic information associated with a patient whose health state is unknown, the trained binary classification model may produce, as output, a prediction that indicates whether the patient has cancer. Said another way, the trained binary classification model may output (i) a first value (e.g., “no” or “0”) in response to a determination that the patient does not have cancer based on an analysis of the genetic information and (ii) a second value (e.g., “yes” or “1”) in response to a determination that the patient has cancer based on an analysis of the genetic information. Because the trained binary classification model is trained to determine the presence of cancer, the trained binary classification model may be referred to as a “cancer detection model” or “cancer yes/no model.”

At block 1408, the processing system 102 can store the trained binary classification model in a storage medium. As part of this process, the processing system 102 may associate contextual information with the trained binary classification model. For example, the processing system 102 may specify, in metadata appended to the trained binary classification model, the cancer types covered by the genetic information that is used as training data. As another example, the processing system 102 may describe the source (e.g., the healthcare system or research institution) of the genetic information used as training data in metadata that is appended to the trained binary classification model.

FIG. 16 includes a flow chart of a method 1600 for training a binary classification model to determine whether an individual is healthy based on an analysis of genetic information. Once again, the method 1600 is described as being performed by the processing system 102 (FIG. 1A) for the purpose of illustration.

At block 1602, the processing system 102 can receive input indicative of a request to train the binary classification model. Generally, this input is provided through an interface that is generated by the processing system 102. Through the interface, an individual (also referred to as an “operator” or “administrator”) may indicate that the binary classification is to be trained. Moreover, the individual may indicate the healthy sample data 1402 (FIG. 14) to be used as training data. For example, the individual may select one or more sources from which to acquire the healthy sample data 1402. As another example, the individual may select the healthy sample data 1402 itself (e.g., by selecting the datasets from among various datasets that are accessible to the processing system 102).

At block 1604, the processing system 102 can obtain multiple datasets of genetic information that are associated with individuals who are suspected of being healthy. Each dataset of the multiple datasets may include genetic information of a corresponding individual that is believed to be healthy. Each dataset of the multiple datasets may be representation of the healthy sample data 1402 available for the corresponding individual. Together, the multiple datasets may be treated as a single dataset by the processing system 102. Accordingly, the processing system 102 may receive, retrieve, or otherwise access a dataset that includes the genetic information of multiple individuals who are suspected of being healthy without any indicators of cancer.

In some implementations, the multiple datasets of genetic information are used for training in their entirety. In other implementations, the processing system 102 can obtain a list of locations for each dataset of the multiple datasets, so as to obtain multiple lists of locations. Each list of locations can be obtained in a manner as discussed above. Because each dataset of genetic information is associated with an individual who is believed to be healthy, the locations will not be expected to include mutations indicative of cancer. Instead, the target locations should include “normal” base pairs and possibly mutations that are not indicative of cancer.

At block 1606, the processing system 102 can provide the multiple datasets of genetic information to an untrained binary classification model as input, so as to produce a trained binary classification model. As mentioned above, the processing system 102 could instead provide a subset of each dataset (e.g., the genetic information corresponding to a list of locations) rather than the entire dataset in some implementations. Upon being applied to genetic information associated with a patient whose health state is unknown, the trained binary classification model may produce, as output, a prediction that indicates whether the patient is healthy. Said another way, the trained binary classification model may output (i) a first value (e.g., “yes” or “1”) in response to a determination that the patient appears to be healthy based on an analysis of the genetic information and (ii) a second value (e.g., “no” or “0”) in response to a determination that the patient appears to not be healthy based on an analysis of the genetic information. Because the trained binary classification model is trained to determine whether a given patient is healthy, the trained binary classification model may be referred to as a “healthy detection model” or “healthy yes/no model.”

At block 1608, the processing system 102 can store the trained binary classification model in a storage medium. As part of this process, the processing system 102 may associate contextual information with the trained binary classification model. For example, the processing system 102 may specify the source of the genetic information (e.g., the healthy sample data 1402) used as training data in metadata that is appended to the trained binary classification model. This metadata could be used, for example, to establish when the trained binary classification model should be retrained or retired (e.g., in favor of a newer version trained using training data of higher quality, with more genetic information, etc.).

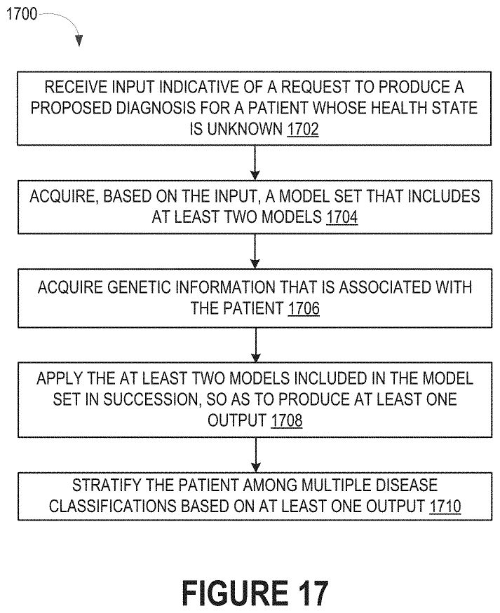

FIG. 17 includes a flow chart of a method 1700 for applying a model set that includes at least two models. At block 1702, a processing system 102 can receive input indicative of a request to produce a proposed diagnosis for a patient whose health state is unknown. Block 1702 of FIG. 17 may be similar to Block 1102 of FIG. 11. Generally, the input is provided through an interface that is generated by the processing system 102. Through the interface, an individual (also referred to as an “operator” or “administrator”) may select or upload genetic information associated with the patient.

At block 1704, the processing system 102 can acquire, based on the input, the model set that includes at least two models. For the purpose of illustration, the model set is described as including (i) a first binary classification model that, when applied to genetic information, produces an output indicative of whether a corresponding individual is healthy, (ii) a second binary classification model that, when applied to genetic information, produces an output indicative of whether a corresponding individual has cancer, and (iii) a multiclass classification model that, when applied to genetic information, produces a series of outputs, each of which is indicative of the likelihood of a corresponding cancer type. The first binary classification model may be trained in accordance with the method 1600 of FIG. 16, the second binary classification model may be trained in accordance with the method 1500 of FIG. 15, and the multiclass model may be trained in accordance with the method 1000 of FIG. 10.

The model set could include different combinations of these models, however. For example, the model set could alternatively include the first binary classification model and multiclass model that are applied in sequence, such that the multiclass model is applied only if the output produced by the first binary classification indicates that the individual is not healthy. As another example, the model set could alternatively include the second binary classification model and multiclass model that are applied in sequence, such that the multiclass model is applied only if the output produced by the second binary classification model indicates that the individual has cancer.

At block 1706, the processing system 102 can acquire genetic information that is associated with the patient. Block 1706 of FIG. 17 may be similar to block 1106 of FIG. 11. As mentioned above, the genetic information could be uploaded through the interface such that it is included in the input. Alternatively, the processing system 102 may acquire the genetic information from a source. The source could be internal to the computing system 100 of which the processing system 102 is a part (e.g., included in memory of the computing system 100), or the source could be external to the computing system 100. For example, the processing system 102 may obtain the genetic information from another computing device (e.g., a sequencing device or computer server). As a specific example, the processing system 102 could retrieve the genetic information from the medical record of the patient that has been made available (e.g., by the healthcare entity that manages the medical record or the patient herself).

At block 1708, the processing system 102 can apply the at least two models included in the model set in succession, so as to produce at least one output. The nature of block 1708 will vary based on which models are included in the model set. Assume, for example, that the model set includes the first binary classification model, second binary classification model, and multiclass model. In such a scenario, those models can be applied in succession, with the second binary classification model and multiclass model being selectively applied based on the outputs produced by the first binary classification model and second binary classification model, respectively. More specifically, the first binary classification model may initially be applied to the genetic information, so as to produce a first output. In the event that the first output indicates the patient is healthy, then the processing system 102 may not take any further action. However, if the first output indicates that the patient is not healthy, then the processing system 102 may apply the second binary classification model, so as to produce a second output. In the event that the second output indicates the patient does not have cancer, then the processing system 102 may not take any further action. However, if the second output indicates that the patient has cancer, then the processing system 102 may apply the multiclass model, so as to produce a third output. As discussed above, the third output may be representative of a set of likelihood values.

At block 1710, the processing system 102 can stratify the patient among multiple disease classifications based on the at least one output produced through implementation of the model set. The multiple disease classifications may vary depending on the desired level of insight to be provided by the processing system 102. One example of possible disease classifications include “healthy” and “cancer.” Another example of possible disease classifications include “healthy,” “Cancer A,” “Cancer B,” . . . , “Cancer N,” where the number of disease classifications is based on the number of cancer types that the multiclass model is trained to identify.

The outputs produced by the model set could also be used by the processing system 102 to stratify patients for examination purposes. Patients that are determined to potentially have a specific type of cancer (e.g., based on the outputs of the multiclass model) may be identified such that examination can be performed more promptly by a healthcare professional, in comparison to patients that are determined to potentially have cancer (e.g., based on the output of the second binary classification model). Similarly, patients that are determined to potentially have cancer (e.g., based on the output of the second binary classification model) may be identified such that examination can be performed more promptly by a healthcare professional, in comparison to patients that are determined to potentially be unhealthy (e.g., based on the output of the first binary classification model). Accordingly, the outputs produced by the first binary classification model, second binary classification model, and multiclass model could be used to inform healthcare systems (and more specifically, healthcare professionals) which patients require examination more urgently. For many types of cancer, the likelihood of survival closely correlates to the stage of discovery—simply put, the earlier that cancer is caught, the more likely that survival is the outcome. By stratifying patients, the processing system 102 can not only act as a diagnostic tool but also as a mechanism for triaging patients in a manner that is most likely to lead to successful outcomes.

Other steps could also be performed. For example, the processing system 102 may store an indication of the disease classification determined for the patient in a digital profile that is maintained for the patient, or the processing system 102 may store the indication in the medical record. As another example, the processing system 102 may determine an appropriate treatment recommendation based on the disease classification. This treatment recommendation could be posted to an interface generated by the processing system 102 for review (e.g., by the individual whose request initiated the method 1700 of FIG. 17). Thus, the processing system 102 may cause display of a visual indicium of the treatment recommendation or another output computed, derived, or otherwise produced by the processing system 102. For example, the processing system 102 may transmit an instruction to display the visual indicium to another computing device across a network, and this other computing device could be associated with the individual whose genetic information is being examined or some other person (e.g., a healthcare professional responsible for overseeing the health of the individual).

### Example of Computing System

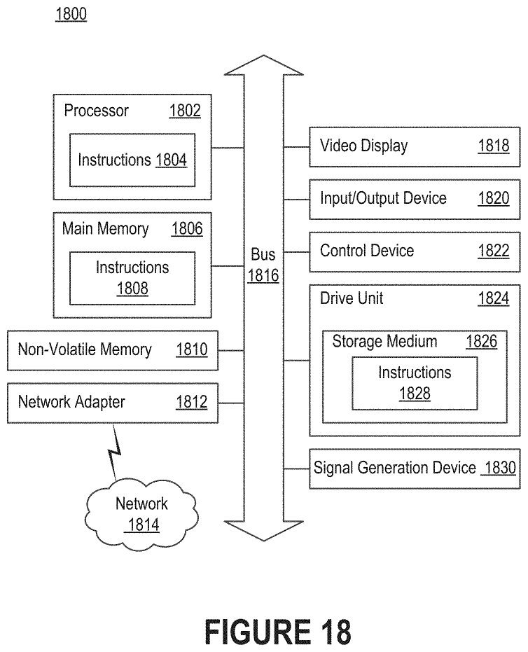

FIG. 18 is a block diagram illustrating an example of a computing system 1800 (e.g., the computing system 100 or a portion thereof, such as the processing system 102) in accordance with one or more implementations of the present technology.

The computing system 1800 may include a processor 1802, main memory 1806, non-volatile memory 1810, network adapter 1812, video display 1818, input/output device 1820, control device 1822 (e.g., a keyboard or pointing device), drive unit 1824 including a storage medium 1826, and signal generation device 1830 that are communicatively connected to a bus 1816. The bus 1816 is illustrated as an abstraction that represents one or more physical buses or point-to-point connections that are connected by appropriate bridges, adapters, or controllers. The bus 1816, therefore, can include a system bus, a Peripheral Component Interconnect (PCI) bus or PCI-Express bus, a HyperTransport or industry standard architecture (ISA) bus, a small computer system interface (SCSI) bus, a universal serial bus (USB), inter-integrated circuit (I2C) bus, or an Institute of Electrical and Electronics Engineers (IEEE) standard 1394 bus (also referred to as “Firewire”).

While the main memory 1806, non-volatile memory 1810, and storage medium 1826 are shown to be a single medium, the terms “machine-readable medium” and “storage medium” should be taken to include a single medium or multiple media (e.g., a centralized/distributed database and/or associated caches and servers) that store one or more sets of instructions 1828. The terms “machine-readable medium” and “storage medium” shall also be taken to include any medium that is capable of storing, encoding, or carrying a set of instructions for execution by the computing system 1800.

In general, the routines executed to implement the embodiments of the disclosure may be implemented as part of an operating system or a specific application, component, program, object, module, or sequence of instructions (collectively referred to as “computer programs”). The computer programs typically comprise one or more instructions (e.g., instructions 1804, 1808, 1828) set at various times in various memory and storage devices in a computing device. When read and executed by the processors 1802, the instruction(s) cause the computing system 1800 to perform operations to execute elements involving the various aspects of the present disclosure.

Further examples of machine- and computer-readable media include recordable-type media, such as volatile memory devices and non-volatile memory devices 1810, removable disks, hard disk drives, and optical disks (e.g., Compact Disk Read-Only Memory (CD-ROMS) and Digital Versatile Disks (DVDs)), and transmission-type media, such as digital and analog communication links.

The network adapter 1812 enables the computing system 1800 to mediate data in a network 1814 with an entity that is external to the computing system 1800 (e.g., between the processing system 102 and the sourcing device 152) through any communication protocol supported by the computing system 1800 and the external entity. The network adapter 1812 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A method comprising:
receiving an input indicative of an instruction to train a multiclass classification model to identify text phrases that are representative of mutations that are diagnostically relevant for multiple cancer types;
accessing a list of locations for each of the multiple cancer types, so as to access multiple lists of locations,
wherein for each of the multiple lists, the locations are representative of different molecular positions at which mutations were discovered through analysis of genetic information of a person known to represent a confirmed instance of a corresponding one of the multiple cancer types;
provide the multiple lists to the multiclass classification model as input, so as to produce a trained multiclass classification model; and
storing the trained multiclass classification model in a storage medium.

2. The method of claim 1, wherein each of the text phrases is representative of a different set of characters, each of which is representative of a nucleotide.

3. The method of claim 1, further comprising:
receiving a second input indicative of a request to analyze genetic information of an individual whose health state is unknown;
applying the trained multiclass classification model to the genetic information, so as to produce an output that includes multiple values, each of which is representative of the likelihood that the individual has a corresponding one of the multiple cancer types; and
stratifying the patient among the multiple cancer types based on an analysis of the multiple values.

4. The method of claim 1, further comprising:
receiving a second input indicative of a request to analyze genetic information of an individual whose health state is unknown;
applying the trained multiclass classification model to the genetic information, so as to produce an output that includes multiple values, each of which is representative of the likelihood that the individual has a corresponding one of the multiple cancer types; and
causing display of a recommendation for further testing of the individual.

5. The method of claim 1, further comprising:
specifying a characteristic of the trained multiclass classification model in metadata that is appended thereto.

6. The method of claim 5, wherein the characteristic is a source from which genetic information used to create the multiple lists of locations was obtained.

7. A non-transitory medium with instructions stored thereon that, when executed by a processor of a computing device, cause the computing device to perform operations comprising:
receiving an input indicative of a request to produce a proposed diagnosis for a patient whose health state is unknown;
accessing, based on the input,
(i) a multiclass classification model, and
(ii) genetic information of the patient;
applying the multiclass classification model to the genetic information of the patient, so as to produce a set of values; and
determining an appropriate diagnosis for the patient based on an analysis of the set of values.

8. The non-transitory medium of claim 7,
wherein the operations further comprise:
applying a binary classification model to the genetic information of the patient, so as to produce an output indicative of whether the patient is healthy;
wherein the multiclass classification model is applied in response to a determination that the output produced by the binary classification model indicates that the patient is not healthy.

9. The non-transitory medium of claim 7,
wherein the operations further comprise:
applying a binary classification model to the genetic information of the patient, so as to produce an output indicative of whether the patient has cancer;
wherein the multiclass classification model is applied in response to a determination that the output produced by the binary classification model indicates that the patient has cancer.

10. The non-transitory medium of claim 7, wherein the input is representative of receipt of the genetic information of the patient from a source external to the computing device.

11. The non-transitory medium of claim 7, wherein the genetic information is representative of sequencing reads of a sample taken from the patient.

12. The non-transitory medium of claim 7, wherein the multiclass classification model is trained to determine the likelihood that the patient has multiple cancer types.

13. The non-transitory medium of claim 12, wherein the set of values includes multiple series of values, each series of values corresponding to a different one of the multiple cancer types.

14. The non-transitory medium of claim 7, wherein the operations further comprise:
populating the set of values into a matrix.

15. The non-transitory medium of claim 14, wherein the appropriate diagnosis is based on a magnitude of values on a diagonal of the matrix.

16. A method comprising:
accessing a multiclass classification model that is trained to distinguish genomic datasets provided as inputs among multiple cancer types;
applying the multiclass classification model to a genomic dataset that includes genetic information of a patient whose health state is unknown, so as to produce a set of values,
wherein each value is indicative of the likelihood that the patient has a corresponding one of the multiple cancer types;
populating the set of values into a data structure;
determining that no values in the data structure exceed a threshold value, and therefore the set of values does not indicate a presence of any of the multiple cancer types;
identifying non-zero values in the set of values for each of the multiple cancer types; and
establishing an appropriate recommendation based on an analysis of the non-zero values.

17. The method of claim 16, wherein the appropriate recommendation specifies a physiological location for further testing, and wherein the physiological location corresponds to the cancer types for which non-zero values were identified.

18. The method of claim 16, wherein the appropriate recommendation specifies how to stratify or prioritize testing of the cancer types for which non-zero values were identified.

19. The method of claim 16, wherein each value produced by the multiclass classification model as output upon being applied to the genomic dataset falls within a range defined by an upper bound and a lower bound, and wherein the threshold value is representative of a midpoint between the upper and lower bounds.

20. The method of claim 16, wherein the data structure is a matrix, and wherein said determining involves an analysis of values on a diagonal of the matrix.
